(19) United States
(12) Patent Application Publication
(10) Pub. No.: US 2026/0298925 A1
(43) Pub. Date: Oct. 1, 2026

(54) SYSTEMS, METHODS, AND COMPOSITIONS FOR THE DETECTION OF MICROBES

(71) Applicant: SD Systems, Inc., Kansas City, MO (US)

(72) Inventors: Elijah SHARPE, Kansas City, MO (US); Darrin CLAWSON, Kansas City, MO (US); Ashley REMIGIO, Kansas City, MO (US); Mitchell LOW, Kansas City, MO (US); Jenny KELLY, Kansas City, MO (US)

(73) Assignee: SD Systems, Inc., Kansas City, MO (US)

(21) Appl. No.: 19/477,667

(22) PCT Filed: Apr. 26, 2024
(86) PCT No.: PCT/US2024/026651

§ 371 Date: Oct. 22, 2025

Related U.S. Application Data
(60) Provisional application No. 63/498,706, filed on Apr. 27, 2023.

(51) Int. Cl. G01N 33/569; G01N 21/27; G01N 21/64; G01N 33/543; G01N 33/58

(52) U.S. Cl. CPC G01N 33/56916; G01N 21/27; G01N 21/6428; G01N 33/54326; G01N 33/54386; G01N 33/582; G01N 33/585; G01N 2021/6439; G01N 2333/245; G01N 2469/10

(57) ABSTRACT

Provided are non-naturally occurring systems, methods, and compositions for the detection of microbes. The disclosure relates to the detection of an antigen specific to a microbe, such as a foodborne, an environment-borne, and a bloodborne bacteria, using capture antibody and moiety, detector antibody and moiety, and a light-emitting particle.

### CROSS REFERENCE TO RELATED APPLICATION

This application claims the benefit of U.S. Provisional Patent Application Ser. No. 63/498,706 filed Apr. 27, 2023. The entire contents of the above-referenced patent application are incorporated by reference in its entirety herein.

### FIELD

This disclosure herein relates to non-naturally occurring systems, methods, and compositions for the detection of microbes such as foodborne, environment borne, and bloodborne bacteria.

### BACKGROUND

Diseases caused by microbes are an important social issue. It presents a widespread and growing threat to human health in both developed and developing countries. Consequently, techniques for the detection of microbes, such as foodborne, environment-borne, and bloodborne bacteria, are needed to prevent the occurrence of human infections. The “gold-standard” method for the detection of microbes is the traditional culture-based bacterial isolation and identification. However, this method is time-consuming and requires expensive and nonportable equipment and specialized facilities, making it inadequate for the rapid detection of microbes to reduce disease occurrence.

Therefore, there is a need for systems, methods, and compositions for the rapid detection and interpretation of microbes.

### SUMMARY

Provided herein are systems, methods, and compositions for the detection of microbes. The disclosure also relates to systems, methods, and compositions for the detection of foodborne, environment-borne, and bloodborne bacteria.

In one aspect, provided herein is a method of detecting an antigen. The method comprises (a) mixing a solution comprising a capture antibody, a detector antibody, and a sample suspected of containing the antigen, wherein: the capture antibody is linked to a capture moiety by a functional group and specifically binds to the antigen in conditions sufficient to form a first complex between the antigen and the capture antibody; and the detector antibody is linked to a detection moiety and specifically binds to the antigen in conditions sufficient to form a second complex between the first complex and the detector antibody, wherein the detection moiety is a light-emitting microsphere; (b) separating unbound detector antibody linked to the detection moiety from the second complex; and (c) detecting the light-emitting microspheres on the second complex by spectroscopy, thereby detecting the antigen.

In another aspect of the disclosure, provided herein is a method of detecting an antigen. The method comprises mixing a solution comprising a capture antibody, a detector antibody, and a sample suspected of containing the antigen, wherein the capture antibody is linked to a capture moiety by a functional group, and the detector antibody is linked to a detection moiety, wherein the detection moiety is a light-emitting microsphere. The method further comprises separating the capture antibody from both the detector antibody and sample. The method further comprises performing a baseline analysis by detecting the light-emitting microspheres from the detector antibody by spectroscopy. The method further comprises mixing and incubating the capture antibody, the detector antibody, and the sample, wherein the capture antibody specifically binds to the antigen in conditions sufficient to form a first complex between the antigen and the capture antibody, and wherein the detector antibody specifically binds to the antigen in conditions sufficient to form a second complex between the first complex and the detector antibody. The method further comprises separating unbound detector antibody from the second complex. The method further comprises performing a final analysis by detecting the light-emitting microspheres on the second complex and/or the light-emitting microspheres from the unbound detector antibody by spectroscopy. The method further comprises comparing the baseline analysis and the final analysis

In another aspect, provided herein is a method of detecting an antigen. The method comprises mixing a solution comprising a capture antibody, a detector antibody, and a sample suspected of containing the antigen, wherein the capture antibody is linked to a capture moiety by a functional group, and the detector antibody is linked to a detection moiety, wherein the detection moiety is a light-emitting microsphere. The method further comprises performing a baseline analysis by detecting the light-emitting microspheres from the detector antibody of the mixture by spectroscopy. The method further comprises incubating the capture antibody, the detector antibody, and the sample, wherein the capture antibody specifically binds to the antigen in conditions sufficient to form a first complex between the antigen and the capture antibody, and wherein the detector antibody specifically binds to the antigen in conditions sufficient to form a second complex between the first complex and the detector antibody. The method further comprises separating unbound detector antibody from the second complex. The method further comprises performing a final analysis by detecting the light-emitting microspheres on the second complex and/or the light-emitting microspheres from the unbound detector antibody by spectroscopy. The method further comprises comparing the baseline analysis and the final analysis.

In some embodiments, the function group is a carboxylic functional group.

In some embodiments, the light-emitting microsphere is a fluorescent particle.

In some embodiments, the fluorescent microsphere comprises a fluorescent dye.

In some embodiments, the fluorescent dye is an Alexa fluorescent dye.

In some embodiments, the fluorescent dye is encapsuled inside the light-emitting microsphere.

In some embodiments, the light-emitting microsphere comprises a polymer encapsulating the fluorescent dye.

In some embodiments, the light-emitting microsphere has an average diameter of between about 20 nm and about 5000 nm, about 100 nm and about 2000 nm, about 100 nm and about 1000 nm, or about 100 nm and about 600 nm.

In some embodiments, the light-emitting microsphere has an average diameter of about 167 nm, about 200 nm, about 300 nm, or about 500 nm.

In some embodiments, the light-emitting microsphere has an excitation wavelength between about 350 nm to about 850 nm.

In some embodiments, the light-emitting microsphere emits light at a wavelength between about 350 nm to about 700 nm.

In some embodiments, the detector antibody is linked to the detection moiety by a second functional group.

In some embodiments, the second functional group is a carboxylic functional group.

In some embodiments, the antigen is located on the surface of a microbe.

In some embodiments, the microbe is a foodborne bacteria, an environment-borne bacteria, or a bloodborne bacteria.

In some embodiments, the microorganism is a bacterium.

In some embodiments, the bacterium is an Escherichia coli or Salmonella spp.

In some embodiments, the Escherichia coli is an Escherichia coliO157: H7 strain.

In some embodiments, the capture moiety comprises a metallic particle, a polymeric particle, or a combination thereof.

In some embodiments, the metallic particle is a magnetic particle.

In some embodiments, the magnetic particle has an average diameter of between about 0.005 μm and about 4.5 μm, about 0.1 μm and about 4.5 μm, about 1.0 μm and about 4.0 μm, or about 2.0 μm and about 3.0 μm.

In some embodiments, the magnetic particle has an average diameter of about 2.8 μm.

In some embodiments, the magnetic particle has an average diameter of between about 0.1 μm and about 2.0 μm, or about 0.1 μm and about 0.5 μm.

In some embodiments, the magnetic particle comprises carboxylic acid groups.

In some embodiments, the method comprises separating unbound detector antibody linked to the detection moiety from the second complex by bounding the second complex to a separation module.

In some embodiments, the second complex is bounded to the separation module using a magnetic field.

In some embodiments, the light-emitting microsphere on the second complex are detected by spectroscopy in solution.

In some embodiments, the detection is qualitative.

In some embodiments, the detection is quantitative.

In some embodiments, the microorganism is present at a concentration of at least about 1.00E+05 CFU/mL, at least about 1.00E+06 CFU/mL, or at least 1.00E+07 CFU/mL.

In some embodiments, the microorganism is present at a concentration of about 1.00E+05 CFU/mL to about 1.00E+10 CFU/mL, about 1.00E+5 CFU/mL to about 1.00E+9 CFU/mL, about 1.00E+5 CFU/mL to about 1.00E+8 CFU/mL, about 1.00E+5 CFU/mL to about 1.00E+7 CFU/mL, or about 1.00E+5 CFU/mL to about 1.00E+6 CFU/mL.

In some embodiments, one or more of the method steps are perform in one or more sample containers, optionally wherein one or more of the method steps are perform in one sample container.

In some embodiments, the sample container has a volume in a range of about 0.5 mL to about 15 mL, about 1.0 mL to about 10, or about 2 mL to about 5 mL.

In some embodiments, the step of incubating comprises heating.

In some embodiments, the one or more of the method steps are perform automatically and/or manually.

In another aspect, provided herein is an apparatus for the detection of an antigen. The apparatus comprise a solution comprising a capture antibody, a detector antibody, and a sample suspected of containing the antigen, wherein: the capture antibody is linked to a capture moiety by a functional group and specifically binds to the antigen in conditions sufficient to form a first complex between the antigen and the capture antibody; and the detector antibody is linked to a detection moiety and specifically binds to the antigen in conditions sufficient to form a second complex between the first complex and the detector antibody, wherein the detection moiety is a light-emitting microsphere. The apparatus comprises a plurality of sample containers, wherein each sample container is configured for: receiving the solution comprising the capture antibody, the detector antibody, and the sample suspected of containing the antigen; receiving illumination at a first wavelength range from a light source; and transmitting radiation from the detection moiety. The apparatus comprise an optical instrument comprising: a plurality of sample container receiving positions, each for receiving a respective one of the plurality of sample containers; the light source adjacent to at least one of the sample containers receiving positions for providing illumination at the first wavelength range and in a first illumination direction through a respective sample container once disposed within the respective sample container receiving position for causing radiation from the detection moiety bound within the detection platform; a carousel with one or more repositionable mirrors for focusing the illumination from the light source; and a photodetector disposed adjacent to the sample container receiving positions for selectively detecting the energy of light radiated from the detection moiety within the respective sample container in response to illumination from the light source and for generating an electrical signal corresponding to the detected energy of the radiated light in response thereto.

In some embodiments, the optical instrument further comprises an amplifier for amplifying the electrical signal output by the photodetector in response to detecting the energy of the radiated light.

In some embodiments, the optical instrument further comprises a digital decoder for converting the detected energy of the radiated light from the respective sample container, represented by the electrical signal output by the photodetector and optionally amplified by the amplifier, to a detectable signal for the detection of the antigen.

In some embodiments, the detectable signal is use as a measure of the concentration of the antigen bound within the second complex, the detected energy of the radiated light being proportional to a measure of detection moiety present in the respective sample container and thus to a measure of antigen concentration bound by the capture antibody.

In another aspect, provided herein is an apparatus for geolocating a source of potential foodborne, environment-borne, and/or bloodborne bacteria. The apparatus comprises a solution comprising a capture antibody, a detector antibody, and a sample suspected of containing the bacteria, wherein: an antigen is located on the surface of the bacteria; the capture antibody is linked to a capture moiety by a functional group and specifically binds to the antigen in conditions sufficient to form a first complex between the antigen and the capture antibody; and the detector antibody is linked to a detection moiety and specifically binds to the antigen in conditions sufficient to form a second complex between the first complex and the detector antibody, wherein the detection moiety is a light-emitting microsphere. The apparatus comprises one or more sample containers, an optical instrument, and a global positioning system (GPS).

These and other aspects of the applicant's teaching are set forth herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Aspects, features, benefits, and advantages of the embodiments described herein will be apparent with regard to the following description, appended claims, and accompanying drawings where:

FIG. 1 is a schematic representation of a direct sandwich enzyme-linked immunosorbent assay (ELISA) according to embodiments of the present teachings.

FIG. 2A is a table of antibodies according to embodiments of the present teachings.

FIG. 2B is a table of antigen preparation items according to embodiments of the present teachings.

FIG. 2C is a table of ELISA preparation items according to embodiments of the present teachings.

FIG. 3A shows images of Escherichia coli O157:H7 bacterial growth plates according to embodiments of the present teachings. Colony counting with sample volume correction yielded a concentration of 9.40E+07 CFU/mL, rounded to 1.00E+08 CFU/mL for ELISA testing. Results were verified by conventional plating and counting of the resultant colony forming units (CFU).

FIG. 3B shows images of K12 bacterial growth plates according to embodiments of the present teachings. Colony counting with sample volume correction yielded a concentration of 3.10E+09 CFU/mL.

FIG. 4A is a table showing the parameters tested in a 1^{st} ELISA screening according to embodiments of the present teachings. The screening was performed with three antibodies and one HRP-conjugate. For each capture antibody, three different antibodies were tested. Heat-inactivated Escherichia coliO157:H7 antigen was selected, and buffer negative/control were assessed.

FIG. 4B is a table showing plate reader results with conditional formatting for the 1^{st} ELISA screening according to embodiments of the present teachings.

FIG. 4C is a table showing plate reader results where highest negative value was identified for the 1^{st} ELISA screening according to embodiments of the present teachings.

FIG. 5A is a table showing the parameters tested in a 2^{nd} ELISA screening according to embodiments of the present teachings.

FIG. 5B is a table showing plate reader results with conditional formatting for the 2^{nd} ELISA screening according to embodiments of the present teachings.

FIG. 5C is a table showing plate reader results where highest negative value was identified for the 2^{nd} ELISA screening according to embodiments of the present teachings.

FIG. 6A is a graph showing the signal obtained from the ELISA sample titration of different Escherichia coli O157:H7 concentrations with CAb-1/Dab-1 according to embodiments of the present teachings.

FIG. 6B is a table showing the signal obtained from the ELISA sample titration of different Escherichia coli O157:H7 concentrations with CAb-1/Dab-1 according to embodiments of the present teachings.

FIG. 7A is a table of antibody pairs that were scored on signal sensitivity as detection and capture antibodies according to embodiments of the present teachings.

FIG. 7B is a table of criteria considered for scoring according to embodiments of the present teachings.

FIG. 8A is a table of the plate format used for pairing matrices according to embodiments of the present teachings.

FIG. 8B is a table showing the results of the ELISA DAb-1 testing with stock (Sarasota) and heat inactivated Escherichia coli O157:H7 according to embodiments of the present teachings.

FIG. 8C is a table showing the results of the ELISA DAb-2 testing with stock (Sarasota) and heat inactivated Escherichia coliO157:H7 according to embodiments of the present teachings.

FIG. 8D is a table showing the results of the ELISA DAb-3 testing with stock (Sarasota) and heat inactivated Escherichia coli O157:H7 according to embodiments of the present teachings.

FIG. 9A is a table showing the results of the ELISA evaluation of Dab-4 at 2 μg/mL according to embodiments of the present teachings.

FIG. 9B is a table showing the results of the ELISA evaluation of Dab-4 at 1 μg/mL according to embodiments of the present teachings.

FIG. 10A is a table showing the results of the ELISA evaluation of Dab-5 at 2.5 μg/mL according to embodiments of the present teachings.

FIG. 10B is a table showing the results of the ELISA evaluation of Dab-6 at 2.5 μg/mL according to embodiments of the present teachings.

FIG. 11 is a table showing conditions that were used to identify optimal antibody pairs according to embodiments of the present teachings.

FIG. 12 is a schematic showing the conjugation process of carboxylic group from magnetic beads according to embodiments of the present teaching.

FIG. 13 is a table of antibody conjugates according to embodiments of the present teaching.

FIG. 14A is a schematic representation of components from a sandwich assay according to embodiments of the present teachings. Illustrated are the capture moiety (center bead), detection moiety (peripheral beads), and detected antigen (sandwiched between the capture antibody located at the surface of the capture moiety and the detector antibody located at the surface of the detection moiety).

FIG. 14B is a schematic representation of a detection moiety according to embodiments of the present teachings. The detection moiety comprises a fluorescent microsphere that can be impregnated with a fluorophore and detector antibodies. The detection moiety is assembled by conjugating the antibodies to the fluorescent microsphere.

FIG. 14C is a schematic representation of a capture moiety according to embodiments of the present teachings. The capture moiety comprises a carboxylated magnetic bead and capture antibodies. The capture moiety is assembled to bind the capture antibodies to the carboxylated magnetic bead. The capture antibody interacts with the carboxyl group of the magnetic bead in order to bind the capture antibodies to the magnetic bead.

FIG. 15 shows images of capture moiety (mGNS AB3) and detector moiety (Dynabeads Ab6) assayed samples according to embodiments of the present teachings.

FIG. 16 shows images of capture moiety (CAb-3 mGNS) and detector moiety (CAb-5 PrA Dynabead) assayed samples for the fluorescence evaluation by UV light at various concentrations of Escherichia coli O 157:H7 according to embodiments of the present teachings.

FIG. 17 is a graph of the mGNS fluorescence testing generated by an optical instrument and algorithmic software according to embodiments of the present teachings.

FIG. 18A is a graph of the mGNS fluorescence testing according to embodiments of the present teachings.

FIG. 18B is a graph of the DynaBead fluorescence testing according to embodiments of the present teachings.

FIG. 19 are images of the capture moiety (mGNS Ab3/Ab6) and the detector moiety (D-bead Ab3/Ab6) assayed samples using fluorescent particles for sample volumes of 150 uL and 500 uL according to embodiments of the present teachings.

FIG. 20A is a graph of the capture moiety (COOH Dyna-Ab6) and detector moiety (Green-Ab6) assayed samples according to embodiments of the present teachings.

FIG. 20B shows images of the capture moiety (COOH Dyna-Ab6) and detector moiety (Green-Ab6) assayed samples according to embodiments of the present teachings.

FIG. 21A is a table summarizing the assay conditions for the evaluation of small-and large-scale conjugations according to embodiments of the present teachings.

FIG. 21B is a graph of the capture moiety (Ab-6) self-paired with the detector moiety according to embodiments of the present teachings. A signal was observed with 1E8 and 1E7 CFU/mL Escherichia coli O157:H7, and best results with large scale conjugates were obtained.

FIG. 22 is a graph of the capture moiety (COOH Dyna-Ab6) and detector moiety (Fluor-Green-Ab6) assay evaluation according to embodiments of the present teachings. 100 uL 1:10 detection moiety dilution yielded the strongest signal and increasing incubation time yielded significantly greater signal.

FIG. 23 is a table summarizing the results from the platform training testing according to embodiments of the present teachings.

FIG. 24 is a table of the 2×2 contingency analysis for the platform training according to embodiments of the present teachings.

FIG. 25 is a flow chart of different steps from the assay according to embodiments of the present teachings. FIG. 25A is a flow chart for a forward assay according to embodiments of the present teaching. FIG. 25B and FIG. 25C are flow charts for reverse assays according to embodiments of the present teaching.

FIG. 26A is a schematic representation of a step from a forward assay according to embodiments of the present teachings. The assay, at this step, contains a capture moiety and detection moiety that are independently or simultaneously suspended in a first mixing buffer.

FIG. 26B is a schematic representation of a step from a forward assay according to embodiments of the present teachings. The assay, at this step, contains a sample which is suspended in the first mixing buffer. The capture moiety, detection moiety, and sample can be added to the first mixing buffer independently and/or simultaneously.

FIG. 26C is a schematic representation of a step from a forward assay according to embodiments of the present teachings. The assay, at this step, is mixed and/or incubated. If the target is present, the capture moiety and the target will bind to form a first complex and the detection moiety will bind to the first complex to form a second complex.

FIG. 26D is a schematic representation of a step from a forward assay according to embodiments of the present teachings. The assay, at this step, uses a separation module to bond the second complex at the bottom of the sample container.

FIG. 26E is a schematic representation of a step from a forward assay according to embodiments of the present teachings. The first mixing buffer optionally comprising unbound capture moiety and unbound detection moiety, at this step, is removed from the sample container and the second complex remains bound to the separation module.

FIG. 26F is a schematic representation of a step from a forward assay according to embodiments of the present teachings. The sample container, at this step, is filled with a second buffer.

FIG. 26G is a schematic representation of a step from a forward assay according to embodiments of the present teachings. The second complex, at this step, is removed from the separation module and suspended in the second buffer for analysis.

FIG. 27A is a schematic representation of a step from a reverse assay according to embodiments of the present teachings. The assay, at this step, contains a capture moiety and detection moiety that are independently or simultaneously suspended in a buffer.

FIG. 27B is a schematic representation of a step from a reverse assay according to embodiments of the present teachings. The assay, at this step, contains a sample which is suspended in the buffer. The capture moiety, detection moiety, and sample can be added to the buffer independently and/or simultaneously, and are mixed and detected as baseline analysis.

FIG. 27C is a schematic representation of a step from a reverse assay according to embodiments of the present teachings. The assay, at this step, is incubated. If the target is present, the capture moiety and the target will bind to form a first complex and the detection moiety will bind to the first complex to form a second complex.

FIG. 27D is a schematic representation of a step from a reverse assay according to embodiments of the present teachings. The assay, at this step, uses a separation module to bond the second complex at the bottom of the sample container. The second complex and/or the unbound detection moiety are detected as final analysis.

FIG. 28A is an image of an angle view of a testing instrument according to embodiments of the present teachings.

FIG. 28B is an image of a side view of a testing instrument according to embodiments of the present teachings.

FIG. 28C is an image of a side view of a testing instrument according to embodiments of the present teachings.

FIG. 28D is an image of a top view of a testing instrument according to embodiments of the present teachings.

FIG. 28E is an image of an angle view of a testing instrument according to embodiments of the present teachings.

FIG. 28F is an image of a top view of a testing instrument according to embodiments of the present teachings.

FIG. 29 is a schematic representation of a testing instrument according to embodiments of the present teachings.

FIG. 30 is a point-of-need application according to embodiments of the present teachings.

FIG. 31 is a test result from the point-of-need application according to embodiments of the present teachings.

FIG. 32 is a schematic representation of architecture of the testing instrument according to embodiments of the present teachings.

FIG. 33 is a schematic representation of the interpretation of the algorithms from the point-of-need application according to embodiments of the present teachings.

FIG. 34 is a schematic representation of data used in the algorithms from the point-of-need application according to embodiments of the present teachings.

### DETAILED DESCRIPTION

It will be appreciated that for clarity, the following disclosure will describe various aspects of embodiments. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment,” “an embodiment,” “an example embodiment,” means that a particular feature, structure, or characteristic described in connection with the embodiment is included in at least one embodiment of the present disclosure. Thus, appearances of the phrases “one embodiment,” “an embodiment,” “an example embodiment,” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the disclosure. For example, in the appended claims, any of the claimed embodiments and/or elements of each claim can be used in any combination.

The disclosure herein relates to systems, methods, and compositions for the detection of microbes from any samples for which microbial contamination is known, suspected, or needs to be determined. Thus, the sample can comprise, for example and without limitation: a fluid; soil; animal and plant products; supplement, food and beverage for human consumption; supplement, food and beverage for animal consumption; a body fluid or body part from an animal; a body fluid or body part from a human; a fluid or part from a vegetable; a fluid or part from a fruit; a fluid or part from a cereal; a surface that has been in contact with environment, human, and animal; and a swab from a surface. In some embodiments, the microbe from the sample is a foodborne bacteria, an environment-borne bacteria, or a bloodborne bacteria. In some embodiments, antigens from the microbe are detected. The disclosure herein also relates to optical system specific for the detection of microbes. The optical system allows for the specific, rapid, simple, and/or sensitive detection of microbes in comparison to the conventional standards of detection. The detection can be qualitative and/or quantitative, and the systems, methods, and compositions can be used in a point-of-testing setting, a point-of-need setting, a point-of-use setting, and/or a point-of-care setting.

The following section headings are for organizational purposes only and are not to be construed as limiting the subject matter described.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. Several definitions are provided below.

The use of the singular forms herein includes the plural unless specifically stated otherwise. As used herein, the singular forms “a,” “an,” and “the” include plural referents unless the context clearly dictates otherwise. Furthermore, use of the term “including” as well as other forms, such as “include,” “includes,” and “included,” is not limiting.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. Numeric ranges are inclusive of the numbers defining the range.

As described herein, any concentration range, percentage range, ratio range or integer range is to be understood to include the value of any integer within the recited range and, when appropriate, fractions thereof (such as one tenth and one hundredth of an integer), unless otherwise indicated.

As used herein, the term “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition.

In some cases, a population of particles may be present. As used herein, the diameter of the particles is an average of a distribution in a particular population.

As used herein, one g/mole is equivalent to one “dalton” (i.e., dalton and g/mol are interchangeable when referring to the molecular weight of a polymer). “Kilodalton” as used herein refers to 1,000 daltons.

As used herein, the terms “fluorophore” and “fluorescent dye,” which are alternatively used in this disclosure, refer to materials than can absorb and/or emit light from the visible region of the spectrum.

As used herein, the term “antibody” refers to an immunoglobulin (Ig) molecule, which generally comprises of four polypeptide chains, two heavy (H) chains and two light (L) chains, or a functional fragment, mutant, variant, or derivative thereof, that retains the epitope binding features of an Ig molecule. Such fragment, mutant, variant, or derivative antibody formats are known in the art. In certain embodiments of a full-length antibody, each heavy chain is comprised of a heavy chain variable region (VH) and a heavy chain constant region (CH). The CH is comprised of three domains, CH1, CH2 and CH3. Each light chain is comprised of a light chain variable region (VL) and a light chain constant region (CL). The CL is comprised of a single CL domain. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG3, IgG4, IgA1 and IgA2), or subclass. Variability exists in the endogenous antibodies between the species.

As used herein, the term “specificity” refers to the ability of a binding protein to selectively bind an antigen.

As used herein, the term “affinity” refers the strength of the interaction between a binding protein and an antigen and is determined by the sequence of the CDRs of the binding protein as well as by the nature of the binding protein and the antigen, such as their size, shape, and/or charge. Binding proteins may be selected for affinities that provide desired therapeutic endpoints while minimizing negative side-effects. Affinity may be measured using methods known to one skilled in the art (e.g., US 2009/0311253, incorporated by reference herein).

As used herein, the term “control” refers to a composition known to not contain an analyte or test substance (“negative control”) or to contain an analyte or test substance (“positive control”). A positive control can comprise a known concentration of an analyte or test substance. A “positive control” can be used to establish assay performance characteristics and is a useful indicator of the integrity of reagents (e.g., analytes or test substances). “Control,” “positive control,” and “calibrator” may also be used interchangeably herein to refer to a composition comprising a known concentration of an analyte or test substance.

As used herein, the term “simulated control” refers to a positive, negative, calibrator, or reference determined through simulation rather than using a known positive or negative control. The simulated control is enabled by training and using algorithms and historical data and can be applied virtually. The use of a simulated control instead of a regular control provides numerous advantages. For instance, food companies generally do not want to do diagnostic testing on-site because most assays require a positive control, and food companies prefer to avoid introducing a known positive to their facilities. The use of a simulated control solves this issue since the control is simulated.

As used herein, the term “Fc region” defines the C-terminal region of an immunoglobulin heavy chain, which may be detached from the variable region of the immunoglobulin by papain digestion of an intact immunoglobulin. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (e.g., U.S. Pat. Nos. 5,648,260 and 5,624,821, incorporated herein by reference).

As used herein, the term “antigen binding portion” or “antigen binding site” or “target binding site” of a binding protein means one or more fragments of a binding protein (e.g., an antibody or receptor), such as an immunoglobulin variable domain (e.g., VH or VL), that retain the ability to specifically bind to an antigen or target. The antigen binding portion of a binding protein can be performed by fragments of a full-length antibody, as well as bispecific, dual specific, or multi-specific formats; specifically binding to two or more antigens. Examples of binding fragments encompassed within the term “antigen binding portion” of an binding protein include (i) an Fab fragment, a monovalent fragment consisting of the VL, VH, CL and CH1 domains; (ii) an F(ab′)2 fragment, a bivalent fragment comprising two Fab fragments linked by a disulfide bridge at the hinge region; (iii) an Fd fragment consisting of the VH and CHI domains; (iv) an Fv fragment consisting of the VL and VH domains of a single arm of an antibody, (v) a dAb fragment, which comprises a single variable domain; and (vi) an isolated complementarity determining region (CDR). Furthermore, although the VH and VL of the Fv, which are encoded by separate genes, can be joined using recombinant methods by a synthetic linker that enables them to be made as a single protein chain in which the VH and VL regions pair to form monovalent molecules (known as single chain Fv (scFv). Such scFvs are also encompassed within the term “antigen binding portion” as are other forms of single chain antibodies, such as diabodies and “linear antibodies” comprising a pair of tandem Fv segments (VH—CH1—VH—CH1) which, together with complementary light chain polypeptides, form a pair of antigen binding sites. Not every amino acid of an antigen binding portion may bind to an antigen. For example, variable domains of an antibody comprise both complementarity determining regions (CDRs) and framework regions (FRs).

As used herein, the term “CDR” means a complementarity determining region within an immunoglobulin variable region sequence. There are three CDRs in each of the variable regions of the heavy chain and the light chain, which are designated CDR1, CDR2 and CDR3, for each of the heavy and light chain variable regions. The term “CDR set” refers to a group of three CDRs that occur in a single variable region capable of binding the antigen. The exact boundaries of these CDRs have been defined differently according to different systems. The system described by Kabat (Kabat et al. (1971) Ann. NY Acad. Sci. 190:382-391; Kabat et al. (1987) Sequences of Proteins of Immunological Interest, Fourth Edition. US Govt. Printing Off. No. 165-492; Kabat et al. (1991) Sequences of Proteins of Immunological Interest, Fifth Edition. NIH Publication No. 91-3242 incorporated herein by reference) not only provides an unambiguous residue numbering system applicable to any variable region of an antibody, but also provides precise residue boundaries defining the three CDRs. These CDRs may be referred to as Kabat CDRs. The terms “Kabat numbering,” “Kabat definitions” and “Kabat labeling” are used interchangeably herein to refer to a system of numbering amino acid residues that are more variable (e.g., hypervariable) than other amino acid residues in the heavy and light chain variable regions of an antibody. Chothia and coworkers (Chothia and Lesk (1987) J. Mol. Biol. 196:901-917; Chothia et al. (1989) Nature 342:877-883) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems.

### Capture and Detector Moieties for Detection of Microbes

Any microbe for which suitable capture and detection methods exist can be used in the systems, methods, and compositions provided herein. In some embodiments, the microbe is a pathogen. Pathogens comprises any eukaryotic or prokaryotic organism known to or suspected of causing disease in a host such as in human and animal.

In one embodiment, the pathogen is Escherichia coli O157:H7. These bacteria are among the major causative agents for foodborne illnesses, which can cause diseases when present in food or water even at as low bacterial numbers as about 50 colony-forming unit (CFU), where the presence of a single CFU in any tested food sample denotes that at least a single living organism is present in the sample. Contaminated ground beef is the most common vehicle for Escherichia coli O157:H7 outbreaks. Beef products may become contaminated during slaughter, and the process of grinding beef may transfer pathogens from the surface of the meat to its interior. Therefore, if ground beef is incompletely cooked, the bacteria can survive. Escherichia coli O157:H7 is an enterohemorrhagic strain capable of inducing diseases in humans through production of Shiga toxins. It has been reported to be associated with several outbreaks of hemorrhagic colitis, which can proceed to life-threatening hemolytic uremic syndrome (HUS), particularly in undiagnosed or untreated cases. The incidence rate of Escherichia coli O157:H7 infection is estimated to be approximately 75,000 cases per year in the United States.

Another suitable pathogen for use with the systems, methods, and compositions provided herein is Salmonella spp., which is the most common cause of meat-associated food borne illness in the United States. Suitable strains comprise, e.g., Salmonella agona; Salmonella anatum; Salmonella enteritidis; Salmonella havana; Salmonella krefeld; Salmonella lilee; Salmonella melegredis; Salmonella montevideo; Salmonella munster; Salmonella newport; Salmonella saintpaul; Salmonella schwarzengrund; Salmonella tennessee; Salmonella typhimurium or Salmonella worthington.

In some embodiments, the pathogen is a bacterial pathogen such as a foodborne pathogen. In some embodiment, the bacterial foodborne pathogen is a member of the Escherichia coli or Salmonella genus. In certain embodiments, the Salmonella species is Salmonella enterica. Other bacterial foodborne pathogens comprise Campylobacter, Clostridium botulinum, Listeria monocytogenes, Staphylococcus aureus, Shigella, and Vibrio vulnificus.

In some embodiments, the pathogen is a viral pathogen such as a foodborne pathogen. In some embodiments, the viral foodborne pathogens comprise norovirus, hepatitis A virus, and rotovirus. Fungal foodborne pathogens comprise those that produce mycotoxins. In certain embodiments, the mycotoxins are aflatoxins. Fungal genera comprise Aspergillus, Penicillium, Paecilomyces and Fusarium. Protist foodborne pathogens comprise Toxoplasma gondii, Cryptosporidium spp. Giardia intestinalis and Cyclospora cayetanensis. Other parasites comprise round worms and tapeworms.

The microbe described herein can be detected by detecting one or more antigens specific to the microbe. The antigen can be coupled and/or not coupled with the microbe. The antigen can be at the surface and/or within the microbe. The antigen can be expressed by one or more microbes. According to some embodiments, the microbes are bacteria, protists, fungi, viruses, or other parasites. According to some embodiments, the antigen is expressed on the surface of the microbe.

The method of detecting the antigen can comprise: (a) mixing a solution comprising a capture antibody, a detector antibody, and a sample suspected of containing the antigen, wherein: the capture antibody is linked to a capture moiety by a functional group and specifically binds to the antigen in conditions sufficient to form a first complex between the antigen and the capture antibody; and the detector antibody is linked to a detection moiety and specifically binds to the antigen in conditions sufficient to form a second complex between the first complex and the detector antibody, wherein the detection moiety is a light-emitting particle; (b) separating unbound detector antibody linked to the detection moiety from the second complex; and/or (c) detecting the light-emitting particles on the second complex by spectroscopy, thereby detecting the antigen. Optionally, the unbound detector antibody linked to the detection moiety is separated from the second complex by bounding the second complex to a separation module, optionally wherein the second complex is bounded to the separation module using a magnetic field. The light-emitting particles on the second complex can be detected by spectroscopy in solution.

The method of detecting the antigen can comprise: (a) optionally incubating a sample solution suspected of containing the antigen to increase the concentration of the antigen within the sample, wherein the sample solution is incubated for at least about 5 seconds, about 10 seconds, about 30 seconds, about 1 minute, about 2 minutes, about 5 minutes, about 10 minutes, about 30 minutes, about 1 hour, about 2 hours, about 5 hours, about 6 hours, about 12 hours, about 24 hours, about 48 hours, or any range of time that is formed from any two of those values as endpoints; (b) suspending a capture moiety and a detector moiety in a buffer within a sample container; (c) aliquoting the sample solution and transferring the aliquot into the sample container to form a reaction mixture comprising the sample, capture moiety, detector moiety, and buffer, wherein the aliquot has a volume of at least about 10 μL, about 20 μL, about 30 μL, about 40 μL, about 50 μL, about 100 μL, about 150 μL, about 200 μL, about 300 μL, about 400 μL, about 500 μL, or any range of volume that is formed from any two of those values as endpoints; (d) placing the sample container on a magnet, wherein the sample is placed on the magnet for about at least 30 seconds, 1 minute, 2 minutes, 3 minutes, 4 minutes, 5 minutes, 10 minutes, or any range of time that is formed from any two of those values as endpoints; (e) analyzing the reaction mixture using an optical instrument to obtain a first value, wherein the first value is a baseline value; (f) incubating the reaction mixture to allow the capture moiety, detector moiety, and antigen to bind, wherein the reaction mixture is incubated for at least about 1 minute, about 2 minutes, about 5 minutes, about 10 minutes, about 30 minutes, about 1 hour, about 2 hours, or any range of time that is formed from any two of those values as endpoints, optionally wherein the incubation is conducted on a shaker and/or rotator; (g) placing the sample container on a magnet, wherein the sample is placed on the magnet for about at least 30 seconds, 1 minute, 2 minutes, 3 minutes, 4 minutes, 5 minutes, 10 minutes, or any range of time that is formed from any two of those values as endpoints; (h) analyzing the reaction mixture using the optical instrument to obtain a second value; and (i) comparing the first and second values to determine the presence, absence, and/or concentration of the antigen within the sample. The comparison can be performed using a software algorithm that calculates the differences between the first and second value to predict the result.

The method of detecting the antigens can comprise: (a) optionally incubating a sample suspected of containing at least two antigens to increase the concentration of the antigens within the sample, wherein the sample is incubated for at least about 5 seconds, about 10 seconds, about 30 seconds, about 1 minute, about 2 minutes, about 5 minutes, about 10 minutes, about 30 minutes, about 1 hour, about 2 hours, about 5 hours, about 6 hours, about 12 hours, about 24 hours, about 48 hours, or any range of time that is formed from any two of those values as endpoints; (b) suspending at least two capture moieties and at least two detector moieties in a buffer, wherein the capture moiety comprises carboxylated beads coated with antibody specific to the antigen, and the detector moiety comprises carboxylated fluorescence particles coated with antibody specific to the antigen; (c) aliquoting the enriched sample solution and transferring the aliquot into the sample container to form a reaction mixture comprising the sample, capture moiety, detector moiety, and buffer, wherein the aliquot has a volume of at least about 10 μL, about 20 μL, about 30 μL, about 40 μL, about 50 μL, about 100 μL, about 150 μL, about 200 μL, about 300 μL, about 400 μL, about 500 μL, or any range of volume that is formed from any two of those values as endpoints; (d) placing the sample container on a magnet, wherein the sample is placed on the magnet for about at least 30 seconds, 1 minute, 2 minutes, 3 minutes, 4 minutes, 5 minutes, 10 minutes, or any range of time that is formed from any two of those values as endpoints; (e) analyzing the reaction mixture using an optical instrument to obtain a first value, wherein the first value is a baseline value, and wherein the optical instrument can use an LED array to illuminate at 365 nm to illuminate all fluorescence particles from 365-900 nm or can use an LED array to illuminate at multiple wavelengths specific to each fluorescence particle; (f) incubating the reaction mixture to allow the capture moiety, detector moiety, and antigen to bind, wherein the reaction mixture is incubated for at least about 1 minute, about 2 minutes, about 5 minutes, about 10 minutes, about 30 minutes, about 1 hour, about 2 hours, or any range of time that is formed from any two of those values as endpoints, optionally wherein the incubation is conducted on a shaker and/or rotator; (g) placing the sample container on a magnet, wherein the sample is placed on the magnet for about at least 30 seconds, 1 minute, 2 minutes, 3 minutes, 4 minutes, 5 minutes, 10 minutes, or any range of time that is formed from any two of those values as endpoints; (h) analyzing the reaction mixture using the optical instrument to obtain a second value, wherein the optical instrument can use an LED array to illuminate at 365 nm to illuminate all fluorescence particles from 365-900 nm or can use an LED array to illuminate at multiple wavelengths specific to each fluorescence particle; and (i) comparing the first and second values to determine the presence, absence, and/or concentration of the antigen within the sample. The comparison can be performed using a software algorithm that calculates the differences between the first and second value to predict the result. The software can report results for multiple targets within a single sample container.

The capture antibody described herein can specifically bind to a desired antigen to be detected. The capture antibody can be either monoclonal or polyclonal. In certain embodiments, the capture antibody is an IgA antibody, IgD antibody, IgE antibody, IgG antibody, IgM antibody, IgY antibody, or a combination thereof. The capture antibody is typically bound to the substrate according to the embodiments described herein. In other embodiments, the detector antibody is an antigen binding portion of an antibody as described herein.

The capture moiety described herein can comprise a metallic particle, non-metallic particle, a polymeric particle, a dendritic particle, or a combination thereof. The capture moiety can comprise a magnetic particle and/or a non-magnetic particle. The capture moiety can be spherical and/or non-spherical. In some embodiments, the capture moiety has an average diameter of at least about 0.005 μm, about 0.01 μm, about 0.02 μm, about 0.05 μm, about 0.1 μm, about 0.2 μm, about 0.3 μm, about 0.4 μm, about 0.5 μm, about 0.6 μm, about 0.7 μm, about 0.8 μm, about 0.9 μm, about 1.0 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, about 3.0 μm, about 3.1 μm, about 3.2 μm, about 3.3 μm, about 3.4 μm, about 3.5 μm, about 3.6 μm, about 3.7 μm, about 3.8 μm, about 4.0 μm, about 4.1 μm, about 4.2 μm, about 4.3 μm, about 4.4 μm, about 4.5 μm, or any range that is formed from any two of those values as endpoints.

In some embodiments, the magnetic particle is a magnetic bead. In some embodiments, the magnetic particle comprises carboxylic acid groups. In some embodiments, the carboxylic acid groups are located at the surface of magnetic particle. The carboxylic groups allow covalent amide bond formation to antibodies, proteins, peptides, or other biomolecules via primary amine (NH₂) groups. Oligonucleotides, antibodies, or other ligands with amine groups can be coupled to the magnetic particles via carbodimide activation. With their large surface area, uniform size, narrow size distribution and unique surface coating, magnetic particles such as MonoMag Carboxyl Beads exhibit high binding capacity and low non-specific binding of protein or nucleic acids.

The functional group described herein to link the capture antibody to the capture moiety and/or the detector antibody to the detection moiety can be any functional group that can link an antibody to the surface of a moiety. The function group can be an immunoglobulin (Ig)-binding proteins (Protein-A, Protein-G). These proteins specifically bind with immunoglobulin G (IgG) Abs and allow for easier and more precise purification of the microbe. The function group can be a carboxylic group, a nitril group, a phosphorus group, a thiol group, an ester group, or any combination or derivative thereof. In some embodiments, the functional group is a carboxylic group.

The detector antibody described herein can specifically bind to the same antigen as the corresponding capture antibody. In certain embodiments, the detector antibody specifically binds to this antigen at a site distinct from which the corresponding capture antibody binds. In certain embodiments, the detector antibody specifically binds to this antigen at a site not distinct from which the corresponding capture antibody binds. According to some embodiments, the detector antibody is not an IgG antibody and/or does not comprise an Fc region from an IgG antibody. In certain embodiments, the detector antibody is an IgY antibody and/or comprises the Fc region from an IgY antibody. According to other embodiments, the detector antibody is selected from an IgA, IgD, IgE or IgM antibody. In other embodiments, the detector antibody is an antibody fragment as described herein.

The detection moiety described herein is any moiety that allows detection, quantification, and/or quantitation of the presence of the microbe, antigen specific to the microbe, detector antibody, and/or detection moiety. The detection moiety can be a particle, a microsphere, and/or a bead. The particle can be a light-emitting particle such as a fluorescent particle. The microsphere can be a light-emitting microsphere such as a fluorescent microsphere. The bead can be a light-emitting bead such as a fluorescent bead. The particle, microsphere, and/or bead can comprise a fluorescent dye. Exemplary fluorescent dyes include, without limitation, fluorescein (e.g., fluorescenin isothiocyanate (FITC)), lanthanide (e.g., lanthanide phosphor), xanthene derivatives (e.g., fluorescein, rhodamine, Oregon green, eosin, and Texas red), cyanine derivatives (e.g., cyanine, indocarbocyanine, oxacarbocyanine, thiacarbocyanine, and merocyanine), squaraine derivatives and ring-substituted squaraines (e.g., seta and square dyes), squaraine rotaxane derivatives (e.g., see tau dyes), naphthalene derivatives (e.g., dansyl and prodan derivatives), coumarin derivatives, oxadiazole derivatives (e.g., pyridyloxazole, nitrobenzoxadiazole, and benzoxadiazole), anthracene derivatives (e.g., anthraquinones, including DRAQ5, DRAQ7, and CyTRAK orange), pyrene derivatives (e.g., cascade blue), oxazine derivatives (e.g., Nile red, Nile blue, cresyl violet, oxazine 170), acridine derivatives (e.g., proflavin, acridine orange, acridine yellow), arylmethine derivatives (e.g., auramine, crystal violet, malachite green), tetrapyrrole derivatives (e.g., porphin, phthalocyanine, bilirubin), and dipyrromethene derivatives (e.g., BODIPY, aza-BODIPY). Exemplary fluorescent dyes include, without limitation, Alexa Fluor (Invitrogen), CF dye (Biotium), DRAQ and CyTRAK probes (BioStatus), BODIPY (Invitrogen), EverFluor (Setareh Biotech), Bella Fluor (Setareh Biotech), DyLight Fluor (Thermo Scientific, Pierce), Atto and Tracy (Sigma Aldrich), FluoProbes (Interchim), Abberior Dyes (Abberior), DY and MegaStokes Dyes (Dyomics), Sulfo Cy dyes (Cyandye), HiLyte Fluor (AnaSpec), Seta, SeTau and Square Dyes (SETA BioMedicals), Quasar and Cal Fluor dyes (Biosearch Technologies), SureLight Dyes (APC, RPEPerCP, Phycobilisomes) (Columbia Biosciences), APC, APCXL, RPE, BPE (Phyco-Biotech, Greensea, Prozyme, Flogen), and Vio Dyes (Miltenyi Biotec). The fluorescent dye can be encapsulated into the particle, microsphere, and bead using a matrix such as a polymer. Exemplary polymers include, without limitation, polystyrene, polyoxymethylene, polyethylene, and derivatives thereof. In some embodiments, the particle, microsphere, or bead is a europium chelated carboxylate fluorescent particle, microsphere, or bead.

The particle, microsphere, and/or bead can have a diameter of at least about 20 nm, about 50 nm, about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 3000 nm, about 4000 nm, about 5000 nm, or any range that is formed from any two of those values as endpoints. In some embodiments, the particle, microsphere, and/or bead can have an average diameter of between about 100 nm and about 2000 nm. In some embodiments, the particle, microsphere, and/or bead can have an average diameter of between about 100 nm and about 1000 nm. In some embodiments, the particle, microsphere, and/or bead can have an average diameter of between about 100 nm and about 600 nm. In some embodiments, the particle, microsphere, and/or bead can have an average diameter of between about 167 nm and about 500 nm.. In some embodiments, the particle, microsphere, and/or bead can have an average diameter of about 100 nm, about 167 nm, about 200 nm, about 300 nm, about 400 nm, about 500 nm, or about 600 nm.

The detection moiety, e.g., the light-emitting microsphere, can have an excitation wavelength between about 350 nm to about 850 nm, and between about 365 nm to about 470 nm. For example, the detection moiety, e.g., the light-emitting microsphere, can have an excitation wavelength of about 365 nm and about 470 nm. The detection moiety, e.g., the light-emitting microsphere, can emit light at a wavelength between about 350 nm to about 700 nm, and between about 525 nm to about 615 nm. For example, the detection moiety can emit light at a wavelength of about 525 and about 615. In some embodiments, the detection moiety, e.g., the light-emitting microsphere, has an excitation wavelength of about 365 nm and an emission wavelength of about 615. In some embodiments, the detection moiety, e.g., the light-emitting microsphere, has an excitation wavelength of about 470 nm and an emission wavelength of about 525 nm.

Applicant discovered that light-emitting g microspheres, e.g., light-emitting microspheres comprising fluorescent dye, exhibit a number of advantages over other detection moieties for the detection of a target, e.g., an antigen. For instance, light-emitting microspheres, such as those comprising fluorescent dyes, exhibit distinct advantages over conventional fluorescent tags. Unlike traditional dyes that are prone to rapid photobleaching, the encapsulation of fluorescent molecules within the polymeric matrix of microspheres significantly enhances their photostability. This structural configuration shields the fluorescent compounds from direct exposure to external oxidative environments, prolonging their emissive properties. The standardized size and spherical symmetry of these microspheres ensure uniform light scattering and emission, leading to more consistent and reliable signal detection across various conditions. These attributes not only improve the performance in terms of enhanced detection sensitivity and lower limits of quantification but also contribute to more robust and repeatable assay outcomes, crucial in diagnostic applications

Other examples of detection moieties comprise, but are not limited to, the following: quantum dots (QD), horseradish peroxidase (HRP), radioisotopes or radionuclides (e.g., 3H, 14C, 35S, 90Y, 99Tc, 111 In, 125I, 131I, 177Lu, 166Ho, or 153Sm); chromogens, fluorescent labels (e.g., FITC, rhodamine, lanthanide phosphors), enzymatic labels (e.g., luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates. Representative examples of labels commonly employed for immunoassays include moieties that produce light, e.g., acridinium compounds, and moieties that produce fluorescence, e.g., fluorescein. In this regard, the detection moiety itself may not be detectably labeled but may become detectable upon reaction with yet another moiety.

### Apparatus for the Analysis, Algorithmic Prediction, and Geolocalization of Microbe

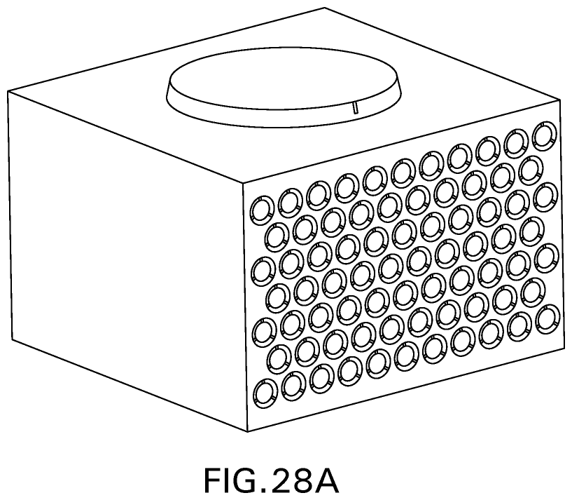

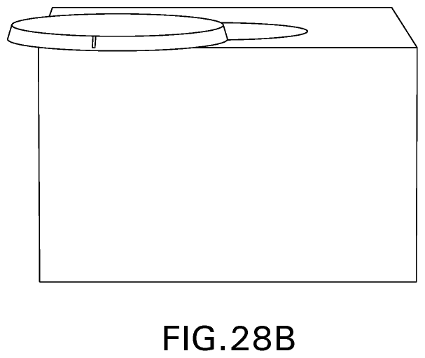

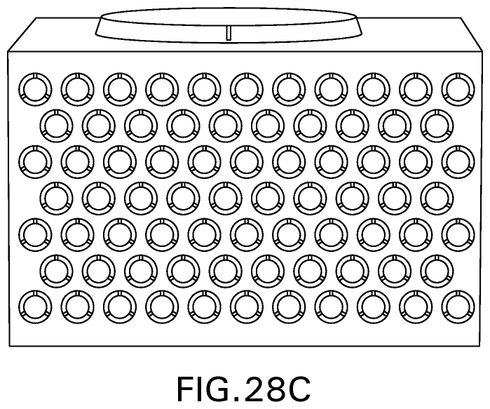

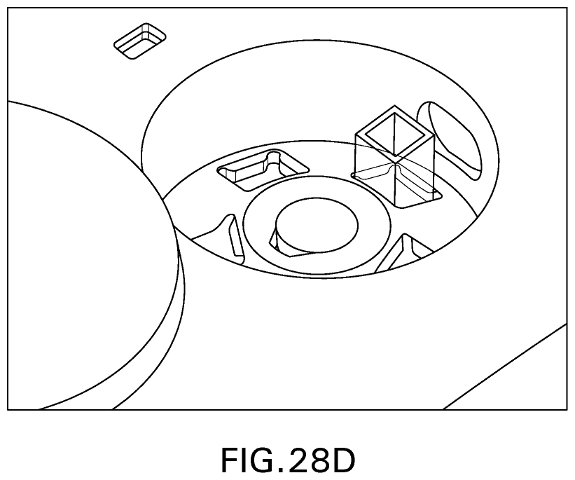

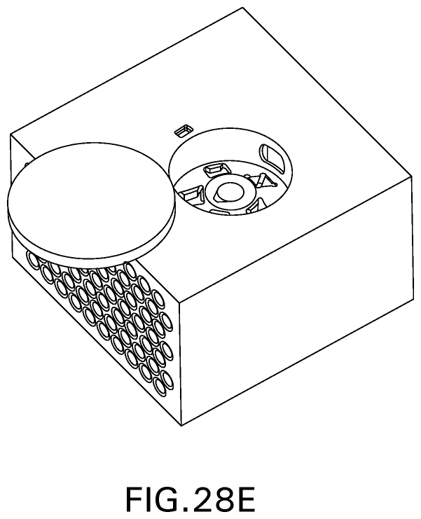

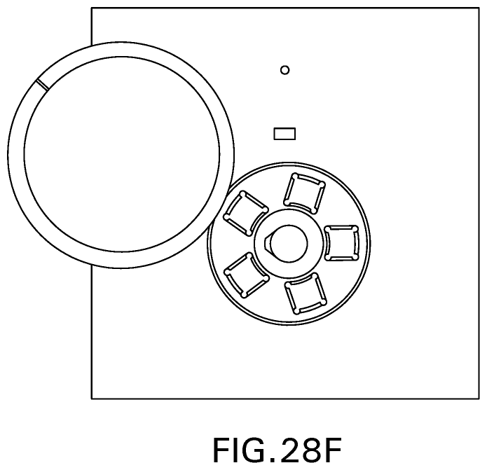

The microbe described herein can be detected by detecting one or more antigens specific to the microbe using an apparatus which can generate a measurable optical signal when the microbe is present in the sample. Examples of apparatus are shown in FIGS. 28A-F and 29.

The apparatuses provided herein to detect the antigen specific to the microbe can comprise a solution comprising a capture antibody, a detector antibody, and a sample suspected of containing the antigen, wherein: the capture antibody is linked to a capture moiety by a functional group and specifically binds to the antigen in conditions sufficient to form a first complex between the antigen and the capture antibody; and the detector antibody is linked to a detection moiety and specifically binds to the antigen in conditions sufficient to form a second complex between the first complex and the detector antibody, wherein the detection moiety is a light-emitting particle.

The apparatuses provided herein can further comprise one or more sample containers, wherein each sample container is configured for performing one or more steps required to detect a target, e.g., an antigen. For example, each sample container is configured for receiving the solution comprising the capture antibody, the detector antibody, and the sample suspected of containing the antigen; mixing a solution; incubating a solution; separating different elements of a solution, e.g., bound and unbound capture antibody, bound and unbound detector antibody, a sample, a first complex, and a second complex; analyzing a solution; receiving illumination from a light source, e.g., receiving illumination at a first, second, third, fourth, and/or fifth wavelength ranges from a light source; and transmitting radiation from the detection moiety. The sample container can have a volume of about 0.5 mL, about 1.0 mL, about 1.5 mL, about 2.0 mL, about 2.5 mL, about 3.0 mL, about 3.5 mL, about 4.0 mL, about 4.5 mL, about 5.0 mL, about 5.5 mL, about 6.0 mL, about 6.5 mL, about 7.0 mL, about 7.5 mL, about 8.0 mL, about 8.5 mL, about 9.0 mL, about 9.5 mL, about 10.0 mL, about 10.5 mL, about 11.0 mL, about 11.5 mL, about 12.0 mL, about 12.5 mL, about 13.0 mL, about 13.5 mL, about 14.0 mL, about 14.5 mL, about 15.0 mL, about 15.5 mL, or any range that is formed from any two of those values as endpoints.

The apparatuses provided herein can further comprise an optical instrument comprising: a plurality of sample container receiving positions, each for receiving a respective one of the plurality of sample containers; the light source adjacent to at least one of the sample container receiving positions for providing illumination at the first wavelength range and in a first illumination direction through a respective sample container once disposed within the respective sample container receiving position for causing radiation from the detection moiety bound within the detection platform; a carousel with one or more repositionable mirrors for focusing the illumination from the light source; and a photodetector disposed adjacent to the sample container receiving positions for selectively detecting the energy of light radiated from the detection moiety within the respective sample container in response to illumination from the light source and for generating an electrical signal corresponding to the detected energy of the radiated light in response thereto. The optical instrument can optionally comprise: an amplifier for amplifying the electrical signal output by the photodetector in response to detecting the energy of the radiated light; and/or a digital decoder for converting the detected energy of the radiated light from the respective sample container, represented by the electrical signal output by the photodetector and amplified by the amplifier, to a interoperable signal for the algorithm to predict the presence or absence of an antigen. The amplifier can amplify or decrease the electrical signal output based on an autonomous integration reading of the cuvette immediately before analysis. Based on the integration reading, logic can be applied to amplify or decrease energy to normalize the radiated light to make it measurable by the optical reader (e.g., to prevent saturation).

The detectable signal to detect the antigen specific to the microbe can be used as a measure of the concentration of the antigen bound within the second complex. The detected energy of the radiated light is proportional to a measure of the detection moiety present in the respective sample container and thus to a measure of antigen concentration bound by the capture antibody. If desired, the apparatus can be used as a benchtop instrument, a point-of-testing instrument, a point-of-use instrument, a point-of-need instrument, and/or a point-of-care instrument.

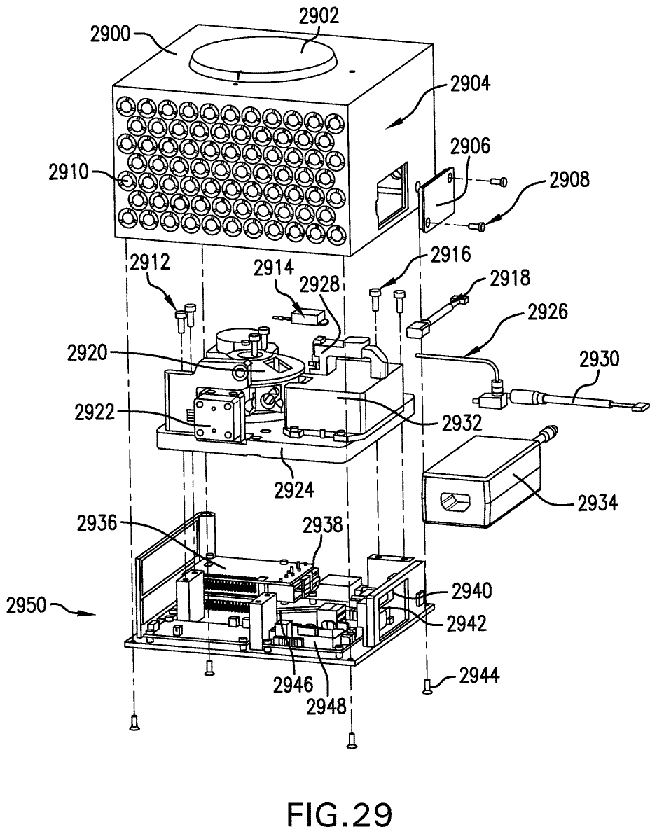

The apparatuses provided herein can comprise the following and other elements. The apparatus can comprise a box 2900 with one dimension having a length of about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, or any range that is formed from any two of those values as endpoints. In some embodiments, the box is about 15 cmΔabout 15 cm×about 15 cm dimensions (FIG. 29). The box can comprise one or more vents 2910 that are designed to maximize air flow, accurately gage the temperature and humidity of its setting, and/or enable a fan-less apparatus. The box can comprise at least one lid 2902 that can be a floated lid that slides clockwise or anti-clockwise to access the inside of the box. The lid can comprise at least one sensor, e.g., an infrared sensor, to sense the status of the lid, e.g., open, and close. The box can comprise a lateral wall 2904 with an access door 2906 fastened to the lateral wall with at least one screw 2908 and designed to give access to the circuit board or ports.

The apparatuses disclosed herein can comprise at least 1 platform, at least 2 platforms, at least 3 platforms, at least 4 platforms, at least 5 platforms, at least 6 platform, or any range that is formed from any two of those values as endpoints. In some embodiments, the apparatus can comprise a top platform 2924 and/or a bottom platform 2950 (FIG. 29), both designed to be inserted into the box 2900.

The apparatuses disclosed herein can be modular such that the carousel, LED array, and/or casing can be switched out depending on the user needs.

The top platform can be designed to host a carousel 2920 (FIG. 29). The carousel can hold at least 1, at least 2, at least 3, at least 4, at least 5, at least 6, at least 7, at least 8, at least 9, or at least 10 cartridges or sample container receiving positions, or any range that is formed from any two of those values as endpoints. The carousel can be designed to perform an analysis cycle for one or more of the cartridges or sample container receiving positions of at least about 1 second, about 3 seconds, about 5 seconds, about 7 seconds, about 9 seconds, about 11 seconds, about 13 seconds, about 15 seconds, about 17 seconds, about 19 seconds, about 21 seconds, about 25 seconds, about 30 seconds, about 60 seconds, about 120 seconds, about 180 seconds, or any range that is formed from any two of those values as endpoints. The carousel can have multiple slits within each slot that allows an LED to illuminate at one position of a cuvette, or multiple positions of a cuvette. Each illumination slit can have repositionable mirrors to concentrate the light source emission. This design advantageously allows to illuminate in one or more slits, enabling control of the sensitivity of detection.

The top platform can be designed to host at least one LED array 2922, that can comprise a blue LED that emits between 365 nm and 450 nm, and/or or a green LED that emits between 500 nm and 550 nm. The LED array can be configured to emit at the beginning of the spectra to excite various fluorophores latter in the spectrum and/or can use an LED to emit at a specific wavelength to excite a specific fluorophore. The top platform can also be designed to host a spectrometer 2932 that can measure emission from the sensor and produce photon flux and digital count values for interpretation. The top platform can be designed to host a camera 2928 that can be used for image recognition, QR imaging, and connectors 2914, 2918, 2926, and 2930. The top platform can be designed to include a separate white light source to illuminate the top portion of a sample container to increase the visibility of the camera. The additional white light source is advantageously designed such that it does not saturate and/or bleach the fluorescent material in the sample container and/or doesn't interfere with LED illumination. The top platform can also be designed to host at least one screw 2912 designed to connect and/or assemble the different elements of the top and/or attached the top platform to the bottom platform.

The bottom platform can be designed to host at least one computer 2936, e.g., a raspberry Pi computer, and at least one computer element 2938 (FIG. 29). The bottom platform can be designed to host wi-fi, cellular, and/or GPS connectors 2946. The bottom platform can be designed to host at least one IoT sensor 2948, e.g., temperature sensor and humidity sensor. The bottom platform can be designed to host at least one port 2942, e.g., a port supporting USB such as USB-C, and/or ethernet, intranet, and/or internet connections. The bottom platform can be designed to host at least one power port 2940 that can be connected to a standard outlet 120V. The apparatus can be designed to go into sleep mode to reduce power consumption and wakes automatically for operation. In some embodiments, no manual power cycling is required.

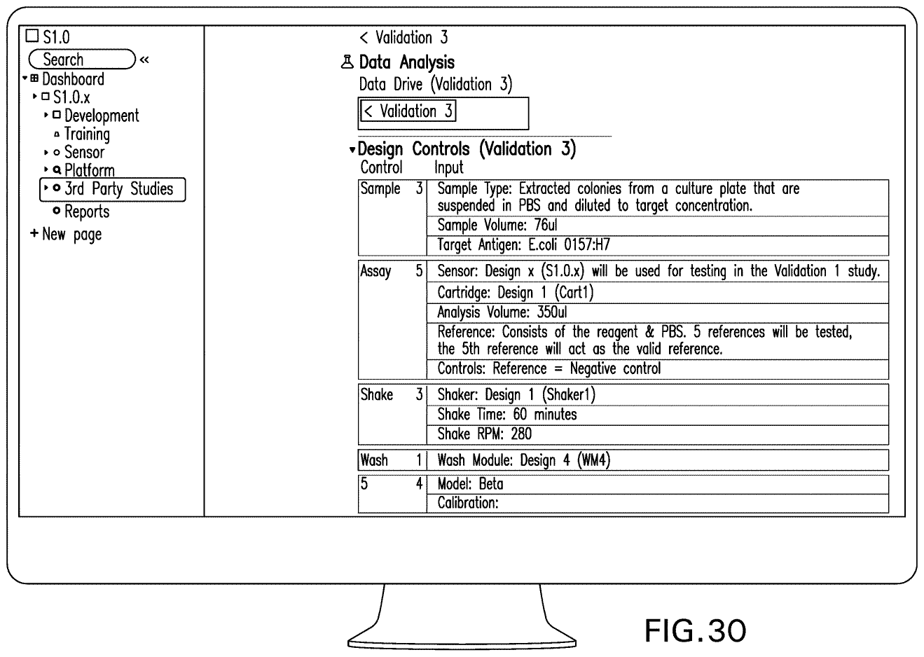

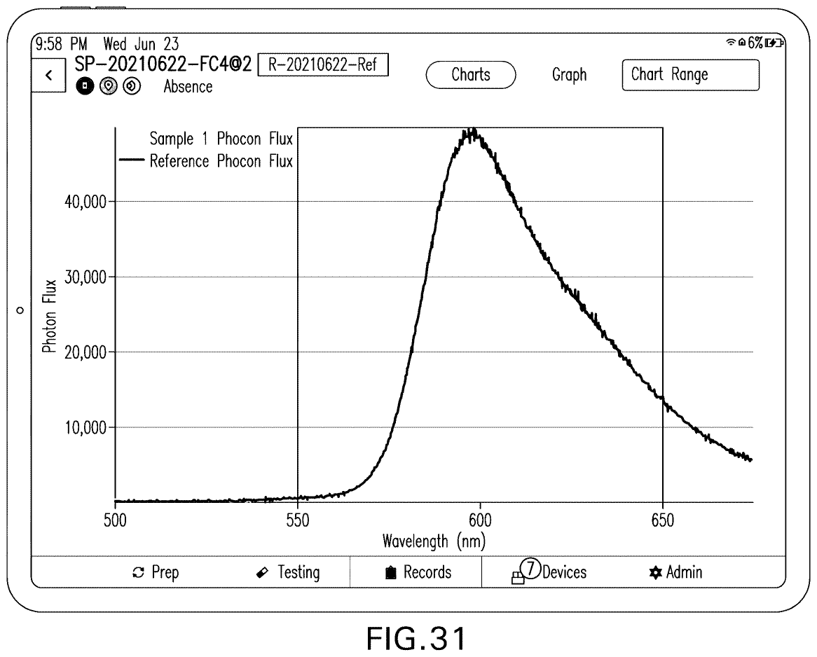

The apparatuses provided herein can be coupled to at least one software, e.g., a computer software and a mobile application (FIGS. 30 and 31). The software can comprise for example, and without limitation, a dashboard platform, a training platform, a monitoring platform, a report platform, a programming platform, a network platform, a development platform, a commerce or marketplace platform, and a 3^{rd} party access platform. The mobile application can comprise for example, without limitation, a preparation application, a testing application, a record application, an administrative application, and a device application.

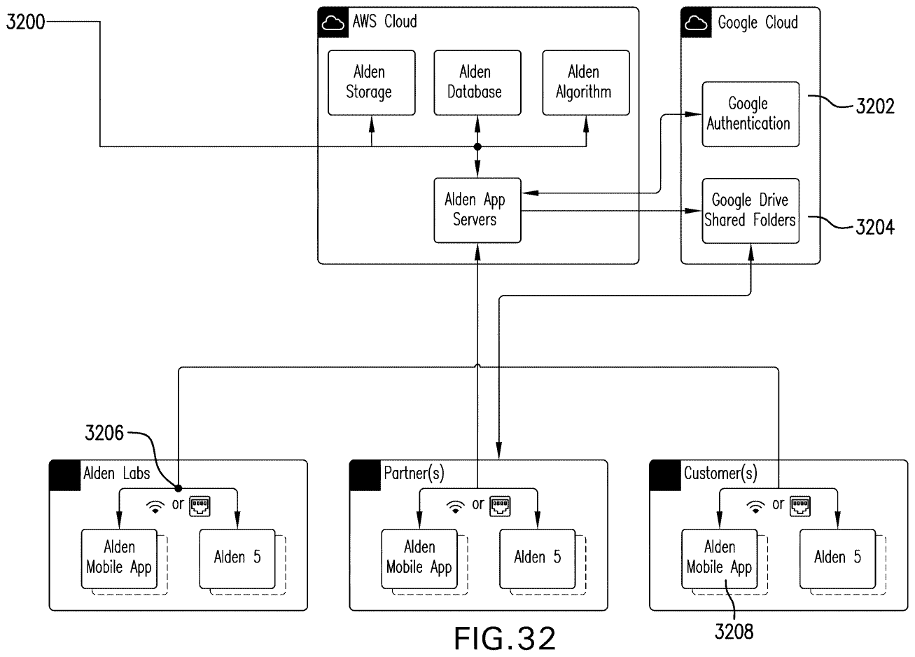

The apparatuses provided herein can be coupled to a computer architecture (FIG. 32). The computer architecture can comprise an AWS Cloud platform comprising a data processing platform 3200. The data processing platform can be used such that analytical test data are exported from the apparatus and stored in a database, exported to an interpreter input sheet, and processed by an algorithm version to provide a result on a software. The computer architecture can comprise network 3206 such as external devices can communicate directly to/from the apparatus. The apparatus and external device optionally can have each separately an internet, intranet, and/or ethernet connection and/or be on the same network. The computer architecture can comprise an authentication application 3202 designed such that each user has an account, e.g., a google account. The computer architecture can comprise a data access application 3204 such that raw data from each analysis can be available shortly after the analysis is completed. The data can be configured to review On an interpreter. The computer architecture can comprise a result platform 3208 where algorithms and machine learning models can be implemented into data processing to provide instant test results.

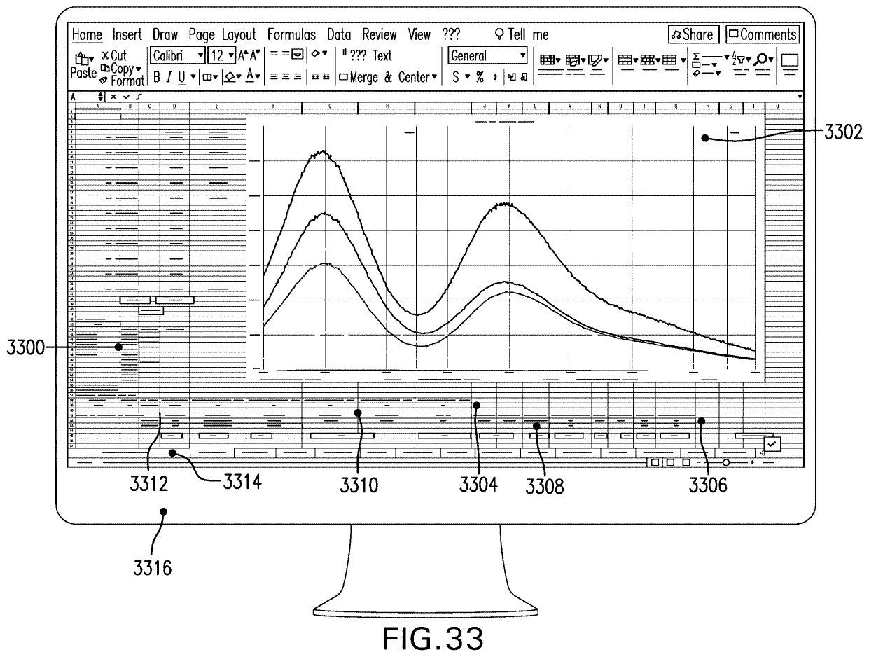

The apparatuses provided herein can use algorithms (FIG. 33). The algorithms can be interfaced using a computer 3316 or mobile device and used to generate graph 3302 (the graph can exhibit wavelengths within the chart range), result table 3300, chart range 3310 (set to view of the spectra by selecting the wavelength), normalized results 3312 (to alter data values to a common scale), input data 3314 (to import data from an input tab), analysis method 3308 to interpret the sample signal (comprising Peak Average, Entire Analysis Range Average, Riemann Sum, and Integration Time Adjusted), comparative analysis (to compare the data of a cuvette at different analysis timepoints to determine difference), smoothing features 3306 (to average set intervals of wavelengths to create a smoothed signal), and analysis ranges 3304.

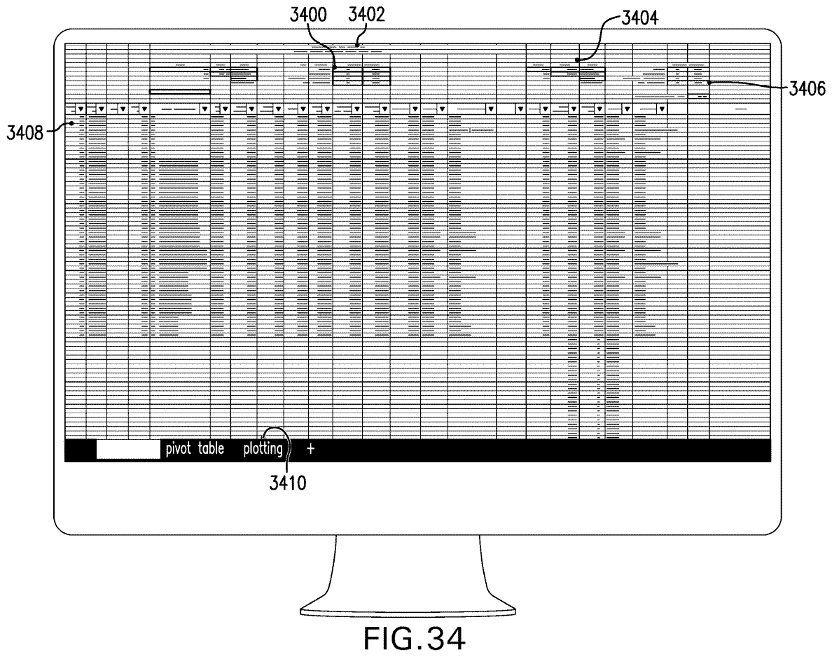

The algorithms can be used for other tasks (FIG. 34). For example, without limitation, a computer display 3402 or mobile display can be used to monitor and perform predictions 3400 with algorithms. The accuracy of the algorithm's prediction is documented in the original correctness table. The table is based on the algorithmic logic applied at the time of analysis. The algorithms can be used for to train data 3408. All training data for a sensor design can be compiled into a summary sheet. The summary sheet applies an algorithm version to labelled data to calculate the algorithms cumulative prediction accuracy. Algorithm modifications can be simulated by adapting algorithm parameters, which may optimize prediction accuracy. The algorithms can be used for plotting 3410. Training data can be configured in a box and whisker plot to interpret statistical significance and clustering of labelled data points. The algorithms can be used to generate and adapt predictions 3406. Adapting the parameters can be used to optimize the algorithms original prediction accuracy.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device

In some embodiments, an optical platform separation (OPS), which is also known as immunomagnetic separation (IMS), is used to isolate target antigens specific to microbes from a fluid known to or suspected of containing the microbe. OPS uses magnetic and/or paramagnetic particles coated with antibodies, e.g., capture antibody, to specifically isolate the target antigens. By applying a magnetic field, the particles with the captured antigen are attracted to the magnet, which allows the rest of the liquid solution to be decanted and/or separated.

In some embodiments, an OPS separation is used to combine the capture of the target antigen with a light detection method to allow for a simultaneous colorimetric quantitation of the target antigen. The capture antibody, which is linked to a capture moiety by a functional group, specifically captures the target antigen. The capture moiety comprises a light-emitting particle and the intensity of the light-emitting particle is proportional to the amount of the detector antibody and thence to the amount of the captured target antigen. Thus, quantification of the resultant colorimetric signal allows for quantification of the target antigen in the sample and thence to the amount of the microbe in the sample.

In some embodiments, the sample is incubated for a period of time under conditions that are conducive to growing the microbe that the sample comprises or is suspected to comprise. For bacteria, a number of bacterial media can be used to enrich a sample for a microbe to increase the chances of detection. In one embodiment, the medium is Tryptic Soy Broth (TSB). In certain embodiments, the TSB comprises novobiocin. Other media comprises LB broth, Terrific broth, M9 minimal medium, and other media suitable for sample enrichment. The sample is grown in media at a desired temperature for a period of time that allows sufficient growth of the microbe to allow its detection. In certain embodiments, the desired temperature is about 25° C., about 26° C., about 27° C., about 28° C., about 29° C., about 30° C., about 31° C., about 32° C., about 33° C., about 34° C., about 35° C., about 36° C., about 38° C., about 39° C., about 40° C., about 41° C., about 42° C., or any range that is formed from any two of those values as endpoints. In certain embodiments, the desired temperature is about “7° C’ In other embodiments, the period of time is from 30 minutes to 24 hours. In other embodiments, the period of time is about 0.5 hour, about 1 hour, about 2 hours, about 3 hours, about 4 hours, about 5 hours, about 6 hours, about 7 hours, about 8 hours, about 9 hours, about 10 hours, about 11 hours, about 12 hours, or any range that is formed from any two of those values as endpoints.

In some embodiments, a detection of antigens is performed with a commercially available photodetector, e.g., a Thorlab Model PDA1001 Newton, New Jersey, to provide a numeric value corresponding to color intensity, which correlates with the amount of the target antigen and thence the amount of the microbe in the tested sample. The light source excitation wavelength and detector bandwidth range are chosen to match sample output wavelength and can be aligned to collect output from multiple sample testing. Detection can alternatively be performed using the apparatus described herein.

In some embodiments, the optical instrument comprises at least one sample container receiving position, at least one light source, at least one carousel, and at least one photodetector. The optical instrument can comprise 1, 2, 3, 4, 5, 6, 7, 8, 9, or 10 sample container receiving position, or any range that is formed from any two of those values as endpoints. The light source is capable of emitting a wavelength of light that is appropriate for exciting or detecting the detection moiety described herein. The detector is capable of detecting light that passes through or from a sample that is held in the sample holder.

In some embodiments, a light source, a photodetector, and a sample container receiving position are arranged to optimize the focus of the light from the light source and alignment in the detection chamber. The sample at the sample container receiving position can be subjected to at least one light source, e.g., one light source, two light sources, three light sources, four light sources, and five light sources, and at least one mirror, e.g., one mirror, two mirrors, three mirrors, four mirrors, and five mirrors, that can be each individually placed to allow the optimized concentration of light that will be projected and collected by the detector.

In some embodiments, the sample holder is motorized so that the samples are moved from a first position where they are placed in the optical instrument and a second position where they are exposed to light from the light source and adjacent the photodetector for detection of radiated illumination. Moving the samples to the second position limits the amount of ambient light that the samples are exposed to when the light source is emitting light. In other embodiments, the second position is located in a temperature-controlled chamber in the optical instrument. According to certain embodiments, this temperature controlled chamber is kept at about 25° C., about 26° C., about 27° C., about 28° C., about 29° C., about 30° C., about 31° C., about 32° C., about 33° C., about 34° C., about 35° C., about 36° C., about 38° C., about 39° C., about 40° C., or any range that is formed from any two of those values as endpoints. According to certain embodiments, this temperature-controlled chamber is kept at about 37° C. In certain embodiments, the sample holders can project a magnetic field to manipulate the magnetic particles that are held in the sample holders. In certain embodiments having a motorized sample holder, the sample holders are oriented in a substantially circular or continuous loop. Sample bearing tubes or cuvettes are introduced proximate to the sample holder pathway, then automatically aspirated and deposited into a cuvette prefilled with first antibody complex. Process steps according to the foregoing disclosure are then performed automatically, including incubation, separation and wash, and prior to exposure to the light source or sources and detection of radiated energy by the photodetector, prior to automatic ejection of the sample cuvette in preparation for a new cuvette.

In some embodiments, the optical instrument comprises one or more digital decoders and transducers for interpreting the results and optionally a digital screen to present the results.

In some embodiments, the optical instrument comprises one or more incubation platforms. In the incubation platform, samples can be kept at the appropriate temperature to enrich the population of microbe to be analyzed. In some embodiments, the optical instrument provides power to maintain the samples at a given temperature. In some embodiment, this temperature is about 25° C., about 26° C., about 27° C., about 28° C., about 29° C., about 30° C., about 31° C., about 32° C., about 33° C., about 34° C., about 35° C., about 36° C., about 38° C., about 39° C., about 40° C., or any range that is formed from any two of those values as endpoints. In some embodiment, this temperature is about 37° C. In some embodiments, the incubation platform is also provided a timing instrument to provide the length of time that samples are incubated.

Also provided herein is also an apparatus for geolocating a source of potential foodborne, environment-borne, or bloodborne bacteria. Such apparatus can comprise: (a) a solution comprising a capture antibody, a detector antibody, and a sample suspected of containing the microorganism, wherein: an antigen is located on the surface of the microorganism; the capture antibody is linked to a capture moiety by a functional group and specifically binds to the antigen in conditions sufficient to form a first complex between the antigen and the capture antibody; and the detector antibody is linked to a detection moiety and specifically binds to the antigen in conditions sufficient to form a second complex between the first complex and the detector antibody, wherein the detection moiety is a light-emitting particle; (b) one or more sample containers; (c) an optical instrument; and (d) a global positioning system (GPS).

Provided herein are also kits that provide in one or more containers some or all of the reagents disclosed herein, e.g., capture and/or detector antibodies disclosed herein, magnetic particles, blocking agents (such as bovine serum albumin or BLOTTO™ mix), as well as reagents for detecting detection moieties of same. The kit may additionally comprise a sealed container. Optionally, an instruction manual for the use of the composition and the information about the composition are comprised in the kit.

### EXAMPLES

While several experimental Examples are contemplated, these Examples are intended non-limiting.

### Example 1

### Materials

The following procedures are used to purify antibodies and conjugate antibodies to capture and detection moieties.

### Antibody Purification

Antibodies are purified using the following procedure:
1. Remove antibody from cold or frozen storage and let sit for 30 minutes.
2. Determine the number of spin columns required.
3. Determine the volume of antibody solution to add to each spin column filter.
4. Calculate the target final volume.
5. Add 450 μL of 1x PBS to each spin column and spin for 5 minutes at 13.8k RCF. Ensure centrifuge is balanced.
6. Remove filter from microcentrifuge tube(s) and dispose of filtrate collected at the bottom of the microcentrifuge tube(s). 50-100 μL of solution should be left in the filter(s).
7. Place filter(s) back inside of the empty microcentrifuge tube(s). Once the antibody solution(s) have equilibrated to room temperature, add calculated antibody solution volume to the spin column filters.
8. Spin filter columns in microcentrifuge tubes for 5 minutes at 13.8k RCF. Ensure centrifuge is balanced.
9. Remove filters from microcentrifuge tubes and collect filtrate from the bottom of the microcentrifuge tubes in a separate, appropriately sized container.
10. Place filters back inside the microcentrifuge tubes. Add 350 μL of 1x PBS to each tube.
11. Repeat steps 8-10 twice more to wash antibody inside the filter a total of three times. Ensure all filtrate is collected into a designated container.
12. After three washes, flip the column filters upside down in the respective, empty microcentrifuge tubes. The filter base will stick out of the microcentrifuge tube.
13. Spin column filters at 1.0k RCF for 5 minutes to transfer purified antibody into microcentrifuge tubes.
14. Acquire a sterile tube that can accommodate the target final volume calculated in step 4. Measure and record the tare weight.
15. Remove filters from microcentrifuge tube and transfer collected purified antibody to tared sterile tube. Measure the total mass of the tube and collected antibody.
16. Calculate the current volume of purified antibody.
17. Calculate volume of 1x PBS to add to collected volume of purified antibody.
18. Add calculated volume of 1x PBS to collected purified antibody. Vortex for 5 seconds to mix.
19. Turn on small-volume spectrophotometer that can measure A280.
20. Take n=3 A280 measurements of the purified antibody solution. Determine average A280 measurement.
21. If the final concentration is significantly lower than expected, perform A280 on collected filtrate to determine if material had leaked through.
22. Label the sterile tube with antibody concentration and date of purification.
23. Store the solution at 2-8° C.

### Example 2

### Antibody Selection and Evaluation by ELISA

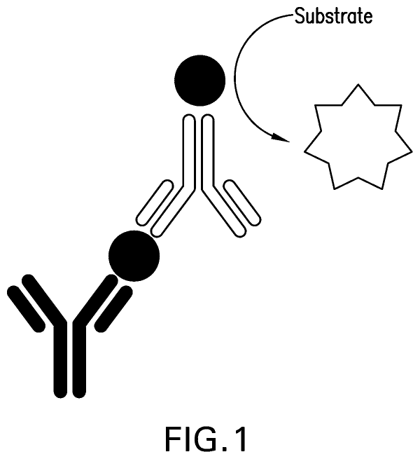

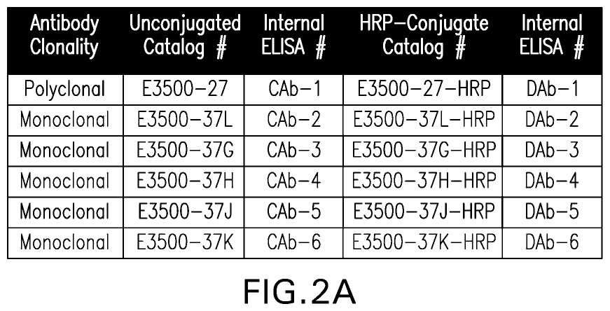

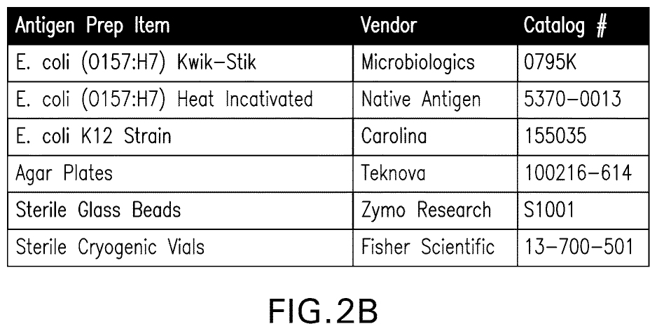

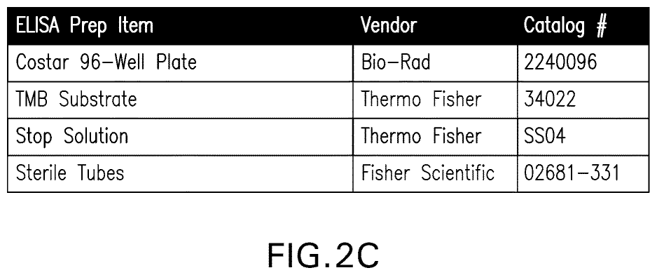

The selection and evaluation by ELISA of antibodies alone and in pair were performed using six unconjugated antibodies and their HRP conjugates tested against Escherichia coli O157:H7 and K12 stocks (FIG. 1, FIG. 2A-C).

### Determination of O157:H7 and K12 Growth CFU/mL

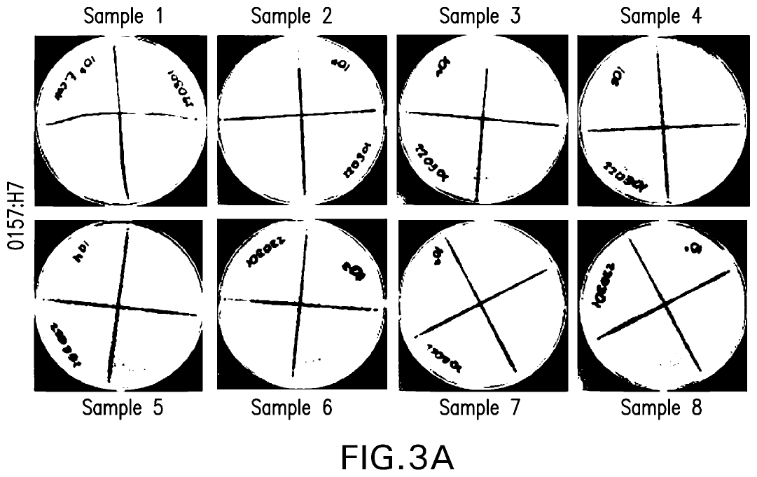

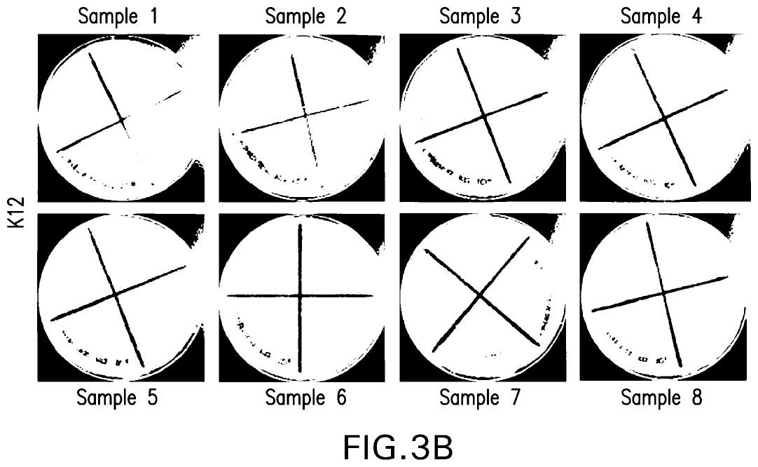

The growth CFU/mL of Escherichia coli O 157:H7 and K12 bacteria were assessed. Non-shiga toxin producing Escherichia coli O157:H7 and wildtype strain K12 were grown. To determine the colony-forming units (CFU) per mL (CFU/mL) of both growths, a serial dilution of each strain was prepared and applied on agar plates for 18-hour incubation at 37° C. During the incubation, the stock was stored at −80° C. to prevent growth. Following the incubation, the colonies formed on agar plates were counted and a back-calculation from the serial dilution was performed to accurately assign the stock CFU/mL. Heat-inactivated Escherichia coli O157:H7 was provided at 1.00E+09 CFU/mL and subsequently aliquoted and stored at −20° C. for individual experiment use. FIGS. 3A-B shows bacterial growth plates for the determination (CFU/mL) of OD 0.77 Escherichia coli O157:H7 stock (FIG. 3A) and K12 stock (FIG. 3B). The serial dilution of stocks in 1x PBS with controlled coverage on agar plates were performed followed by the addition of 100 μL sample to respective plate, incubation at 37° C. for 18-24 hours, and counting of the colonies. Escherichia coli O157:H7 colony counting with sample volume correction yielded a concentration of 9.40E+07 CFU/mL, rounded to 1.00E+08 CFU/mL for ELISA testing. K12 colony counting with sample volume correction yielded a concentration of 3.10E+09 CFU/mL

An antibody selection utilizing a direct-ELISA sandwich assay approach was performed using these cultured samples. To accomplish this, unconjugated antibodies were immobilized on the surface of wells of a high-binding 96-well plate. The target sample was then applied to the wells and incubated for approximately 1 hour to allow specific binding interactions to take place. The HRP-conjugated antibody was added to complete the sandwich, TMB was as the substrate to elicit the signal-producing reaction of HRP. The following screenings were performed accordingly.

### 1^{st} ELISA Screening

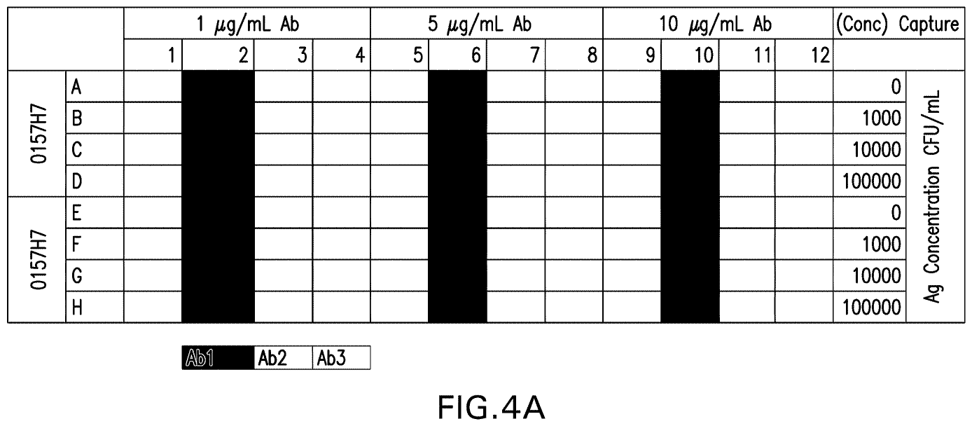

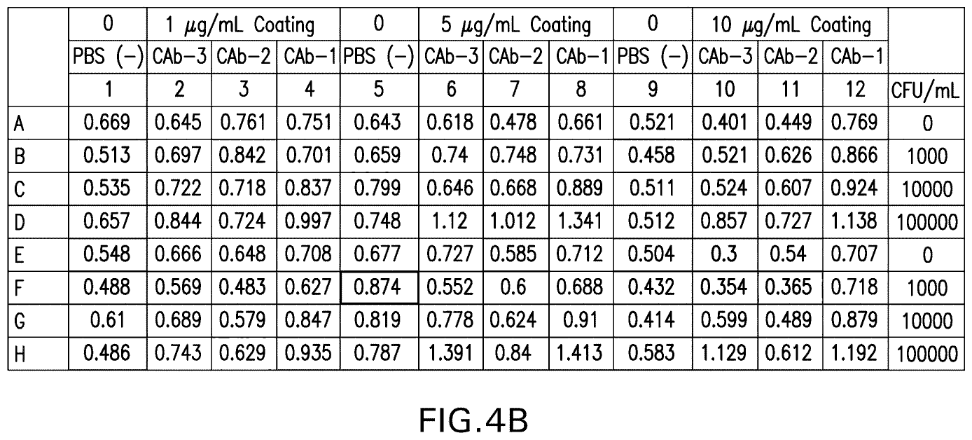

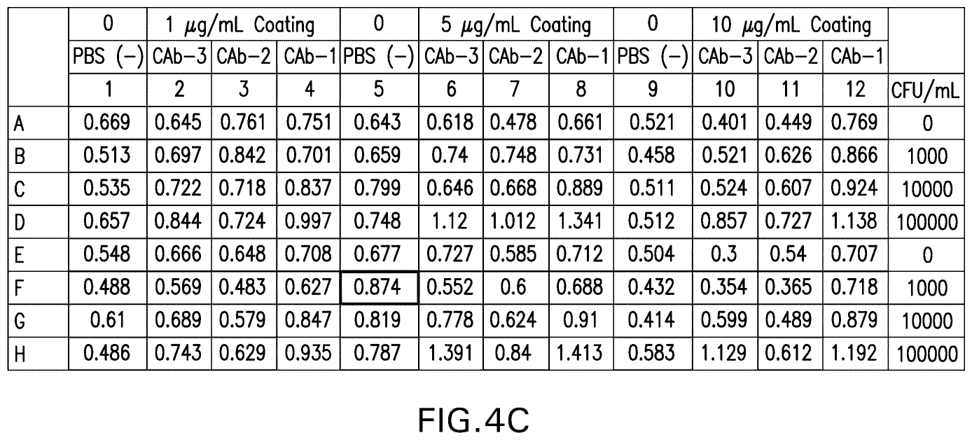

A first ELISA screening utilizing one polyclonal and two monoclonal capture antibodies (cAb-1, cAb-2, and cAb-3) with one polyclonal detector antibody (dAb-1) was performed using the following methods (FIGS. 4A-4C):
Each antibody coated at 3 different concentrations: 1, 5, and 10 μg/mL;
Initial screening of the pairs was performed with heat inactivated Escherichia coli O157:H7 at 4 different concentrations: 0, 1.00E+03, 1.00E+04, and 1.00E+05 CFU/mL; and
1x PBS was utilized as a negative control.

cAb-1 and cAb-3 were both observed to produce a positive signal (above background) with dAb-1 and repeat testing of these pairs was performed to optimize ELISA procedure for future testing while simultaneously reducing background noise with negative samples. Specifically, the results indicate that polyclonal antibody appear to self-pair as the capture (Cab-1) and HRP-tagged-detecting (Dab-1) antibody to achieve readings as low as 10,000 (1.00E+04) CFU/mL at coated concentration of 5 μg/mL and 10 μg/mL. It was also found that Cab-3 also pairs with Dab-1, pairing with Cab-3 concentrations of 5 μg/mL and 10 μg/mL and resulting in a signal at 100,000 (1.00E+05) CFU/mL.

### 2^{nd} ELISA Screening

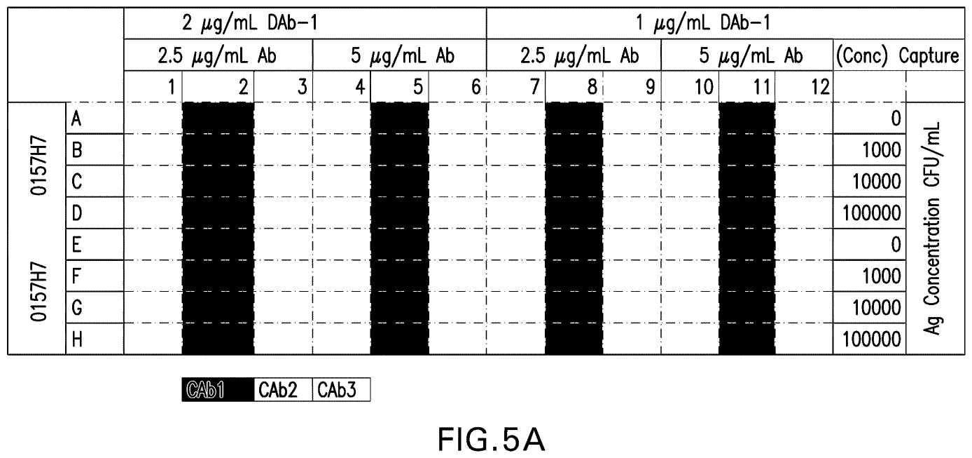

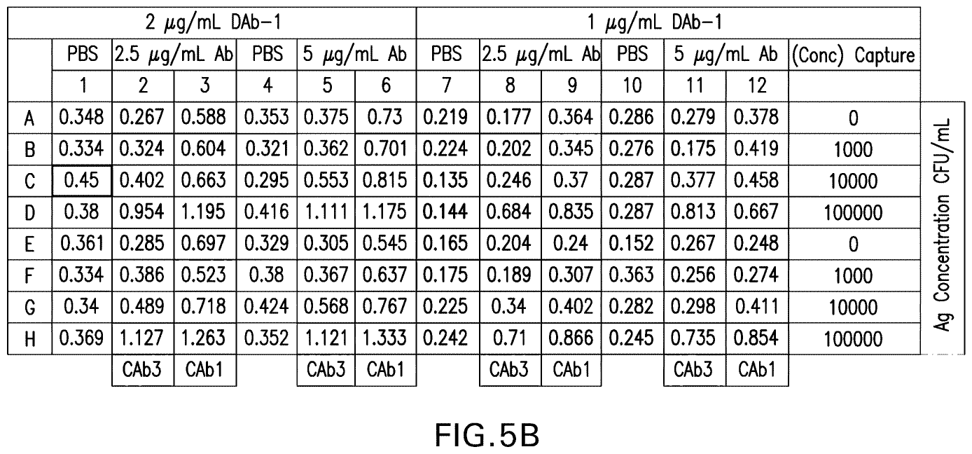

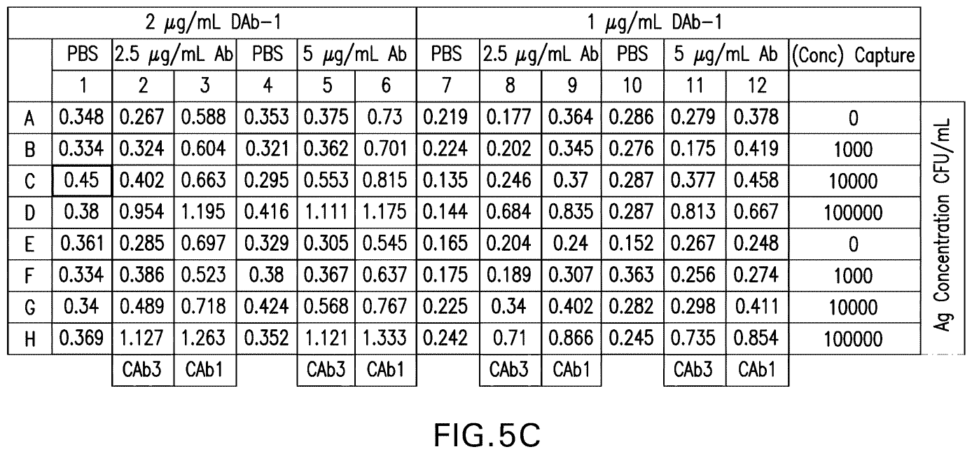

A second ELISA screening that focuses on cAb-1 and cAb-3 was performed using the following methods (FIGS. 5A-C):
Each antibody coated at 2 different concentrations: 2.5 μg/mL, 5 μg/mL;
Antigen (Heat-Inactivated Escherichia coli O157:H7) added at four different concentrations: 0 CFU/mL, 1,000 CFU/mL, 10,000 CFU/mL, 100,000 CFU/mL;
dAb-1 added at two different concentrations: 2 μg/mL, 1 μg/mL; and
Each combination tested in duplicate.

The background noise from the screening was found to be reduced from first pass. Nonspecific signal (absence of antigen) was observed with cAb-1 (self-pairing with dAb-1). Both cAb-1 and cAb-3 were found to pair with dAb-1 at capture concentrations of 2.5 μg/mL and 5.0 μg/mL, and a signal was found to be distinguishable from non-specific at 100,000 CFU/mL. It was observed that the reduction of detector antibody concentration to 1 μg/mL was found to resolve the high non-specific signal (see rows A and E for cAb-1). No significant increase in signal between capture antibody concentrations of 2.5 μg/mL and 5.0 μg/mL was observed, and therefore the conditions to consider in future testing were selected to include 2.5 μg/mL of capture antibody coating concentration and 1 μg/mL of detector antibody concentration.

### ELISA Sample Titration

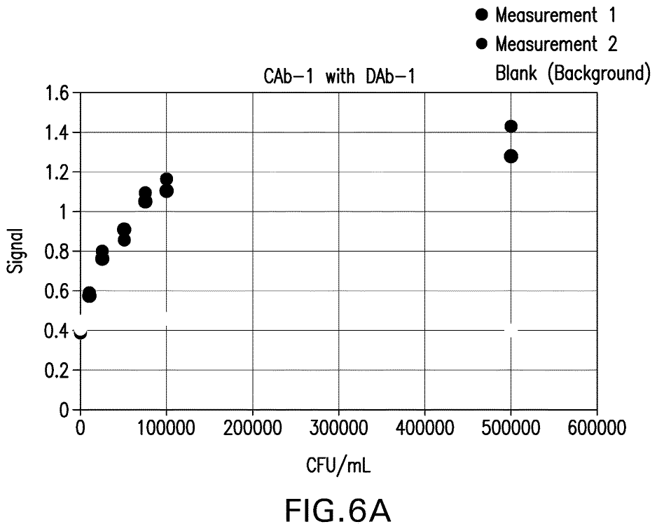

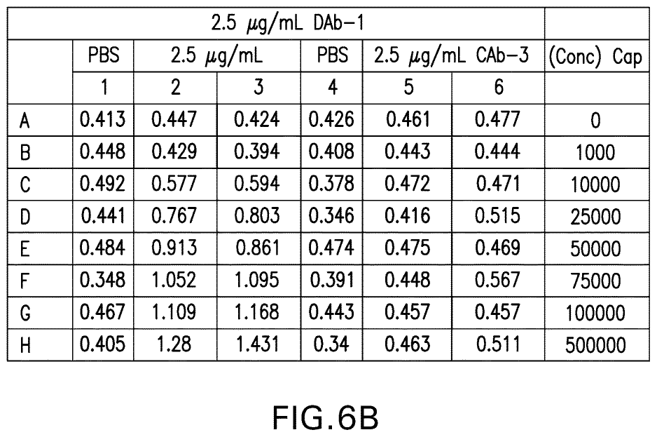

ELISA was performed with a range of antigen (heat-inactivated Escherichia coli O157:H7) concentrations to determine signal trend and establish proof of true pairing signal using the following methods (FIGS. 6A-6B):
Previously established conditions in ELISA optimization; and
Escherichia coli O157:H7 at 0, 1,000, 10,000, 25,000, 50,000, 75,000, 100,000, and 500,000 CFU/mL.

The detector antibody (dAb-1) was prepared at 2.5 μg/mL. The trend with self-pairing to cAb-1 was found to be clearly visible. As antigen concentration increases, the signal was found to begin to plateau. Regarding dAb-1 and cAb-3 pairing, no trend was observed.

Elisa Optimal Ab Pairing Selection (cAb-1, cAB-2, and cAb-3)

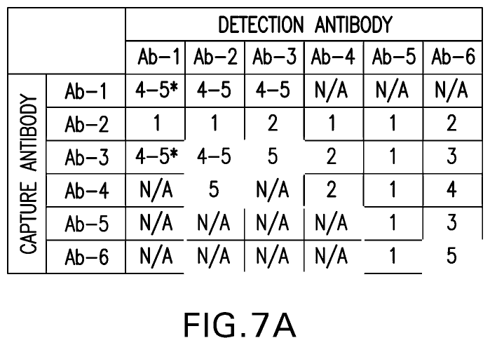

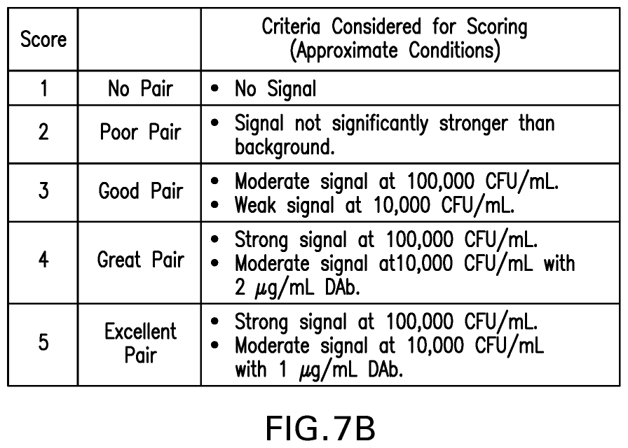

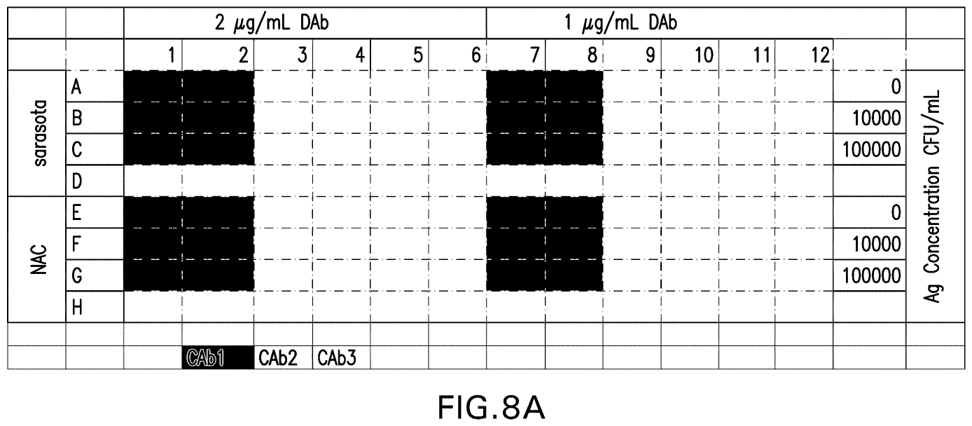

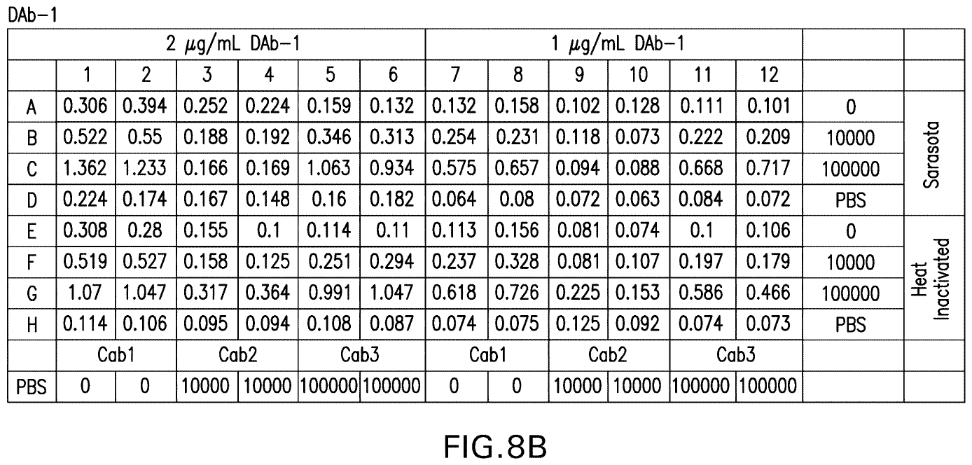

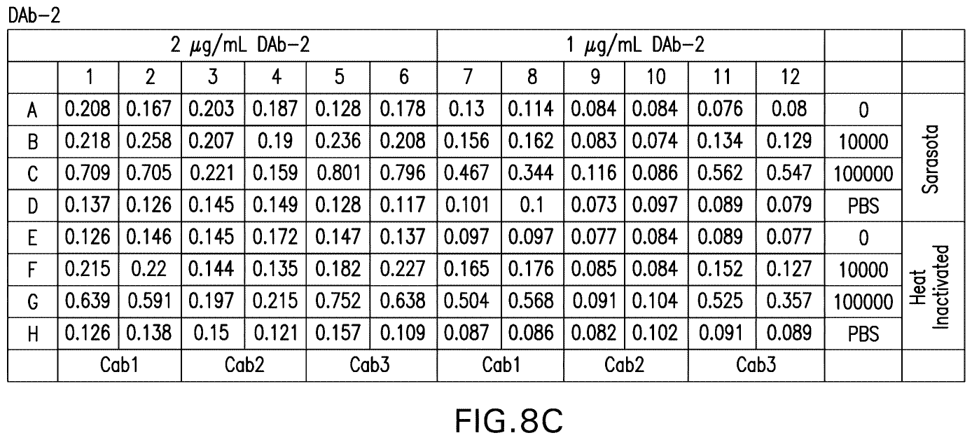

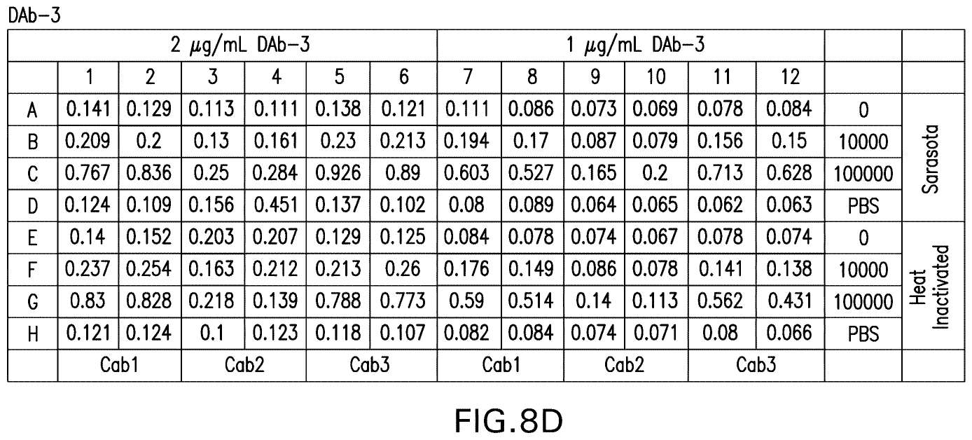

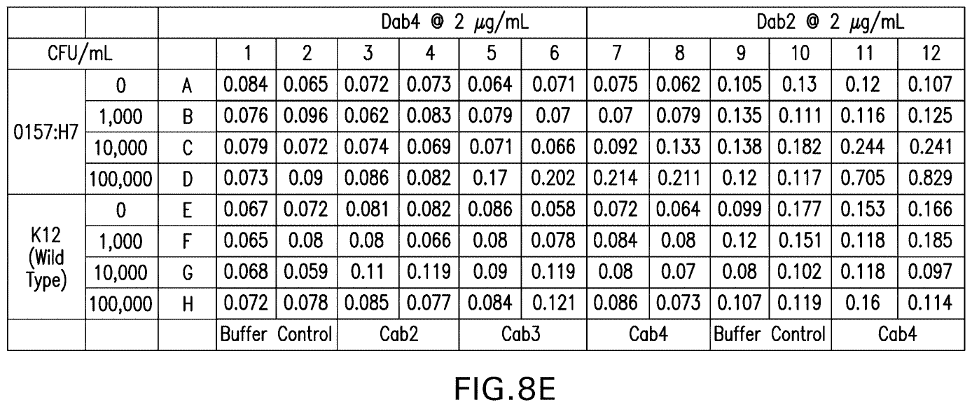

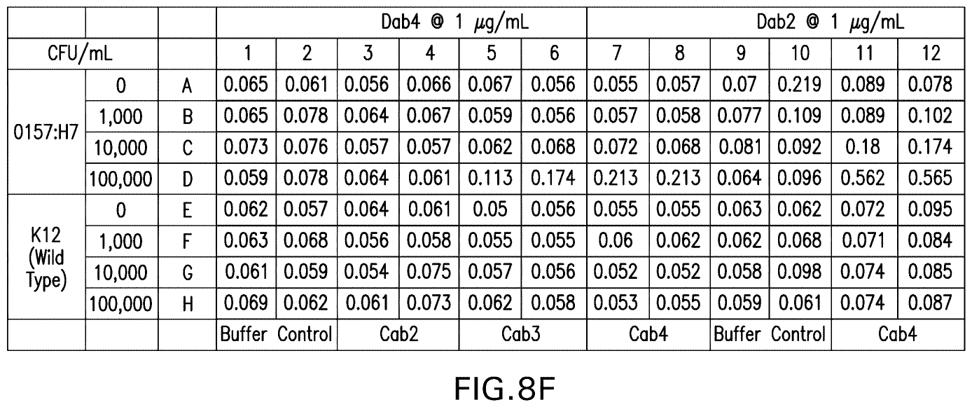

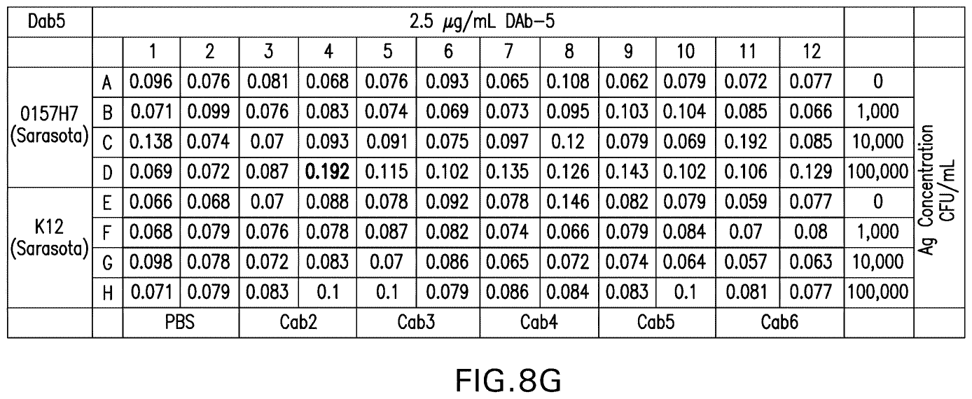

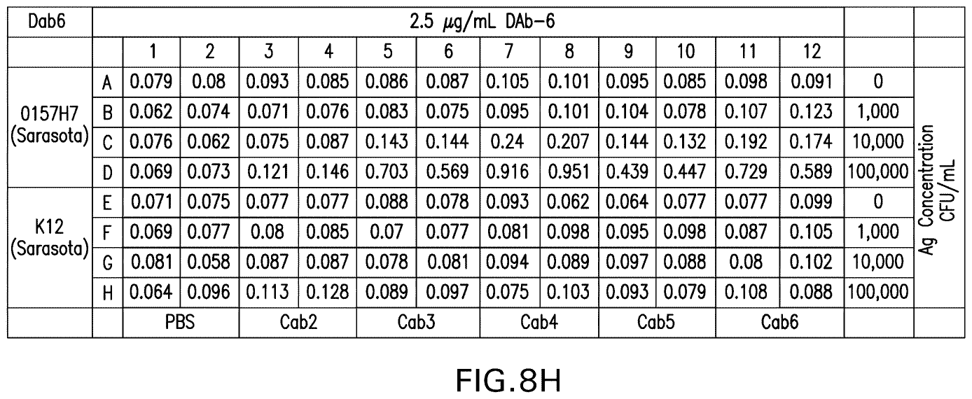

Optimized ELISA testing was done between the Escherichia coli O157:H7 bacteria and the heat-inactivated Escherichia coli O157:H7 (positive samples) using the K12 wildtype as a negative sample. Capture antibodies and detector antibodies were evaluated by ELISA with antibody pairings scored on signal intensity, specificity, and sensitivity (FIGS. 7A-7B). Pairings of detector antibodies dAb-1, dAb-2, and dAb-3 with capture antibodies cAb-1, cAb-2, and cAb-3 were evaluated (FIGS. 8A-8D). The simultaneous evaluation of heat-inactivated Escherichia coli O157:H7 to in-house stock was performed. The following methods were used:
Coating concentration: 2.5 μg/mL.
Detection concentration: 2 g/mL, 1 μg/mL
Antigen concentration: 0, 10,000, 100,000 CFU/mL 1 plate per detector antibody (3 plates total)

For the dAb-1 plate with stock (Sarasota) or heat inactivated Escherichia coli O157:H7 (FIG. 8B), pairings for cAb-1 and cAb-3 and high background noise were observed, and self-pairing (cAb-1/dAb-1) was stronger. For the dAb-2 plate with stock (Sarasota) or heat inactivated Escherichia coli O157:H7 (FIG. 8C), pairing with Cab-1 and Cab-3, lower background noise than Dab-1 plate, and no significant self-pairing were observed. For the Dab-3 plate with stock (Sarasota) or heat inactivated Escherichia coli O157:H7 (FIG. 8D), pairing with Cab-1 and Cab-3, self-pairing, and lowest background noise of three pates evaluated were observed. Overall, signals between stock (Sarasota) and heat inactivated Escherichia coli O157:H7 were found to be similar. Overall, all three detector antibodies were found to exhibit positive pairing signal with cAb-1 and cAb-3, no significant signal was observed with cAb-2, and both Escherichia coli O157:H7 (stock and heat-inactivated) yielded similar results.

ELISA Evaluation of cAb-4 and Dab-4

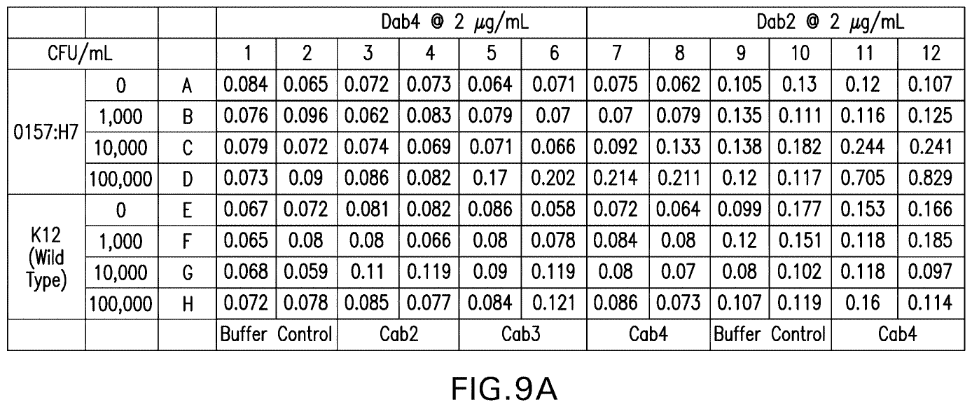

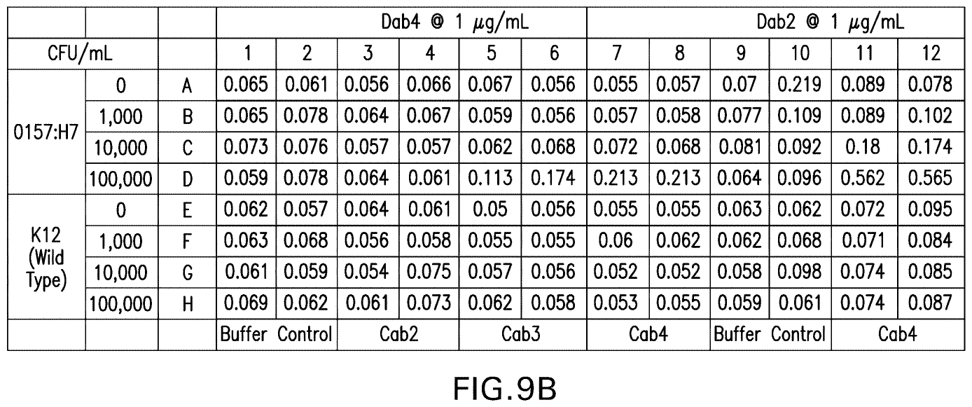

The ELISA evaluation of cAb-4 and dAb-4 was performed (FIGS. 9A-9B). No pairing with cAb-2 and paring with cAb-3 and cAb-4 at 100,000 CFU/mL were observed for dAb-4. Pairing with dAb-2 at 1 μg/mL and 2 μg/mL and initial screening with signal at 10,000 CFU/mL were observed for cAb-4. A low background noise for both plates compared to previous polyclonal plates and no cross reactivity with K12 across the different pairing combinations were observed.

ELISA Evaluation of cAb-5/6 and Dab-5/6

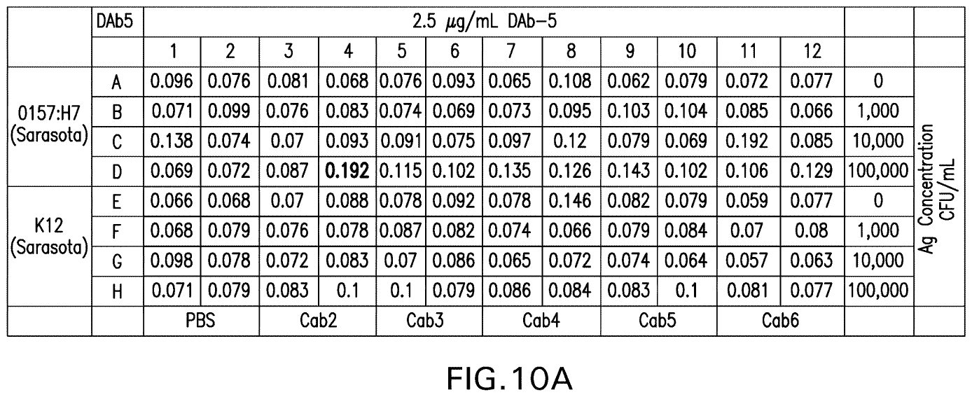

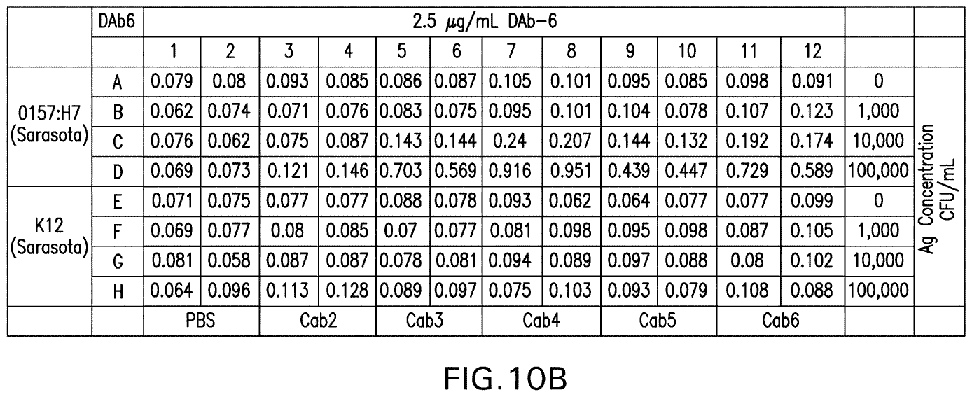

The ELISA evaluation of cAb-5/6 and dAb-5/6 was performed (FIGS. 10A-10B). Antibodies 5 and 6 were screened against capture antibodies cAb-2, cAb-3, cAb-4, cAb-5, and cAb-6 and detector antibodies dAb-5 (FIG. 10A) and dAb-6 (FIG. 10B). It was observed that antibody 5 is a poor detector antibody and a moderate capture antibody with Dab-6, and antibody 6 is a good detector antibody with cAb-3, cAb-4, cAb-5, and cAb-6. Overall, very low background noise and no significant cross reactivity with K12 across the different pairing combinations were observed.

### Final Working Conditions

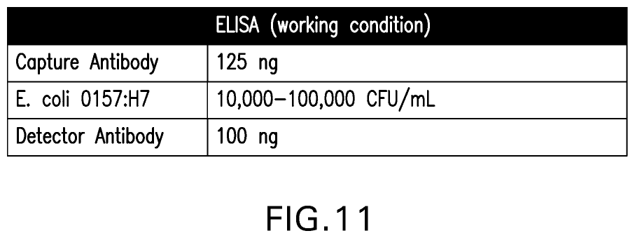

A final working condition for the ELISA was established using 125 ng of capture antibody and 100 ng of detector antibody per test, yielding positive sample detection sensitivity between 1.00E+04 to 1.00E+05 CFU/mL (FIG. 11).

### Example 3

Escherichia coli O157:H7 Assays on a Detection Apparatus

Escherichia coli O157:H7 assays were performed on a Detection Apparatus.

### Material Sourcing and Conjugate Preparation

The assay on the detection apparatus can require a selected capture antibody to be conjugated to the surface of magnetic particles. Protein A beads, such as Protein A dynabeads, are commonly used to conjugate capture antibodies to perform assays. However, the use of protein A surfaces on beads may vary lot-to-lot in performance and/or storage stability. Instead, a variety of alternate magnetic capture particles for use with the detection apparatus were assessed.

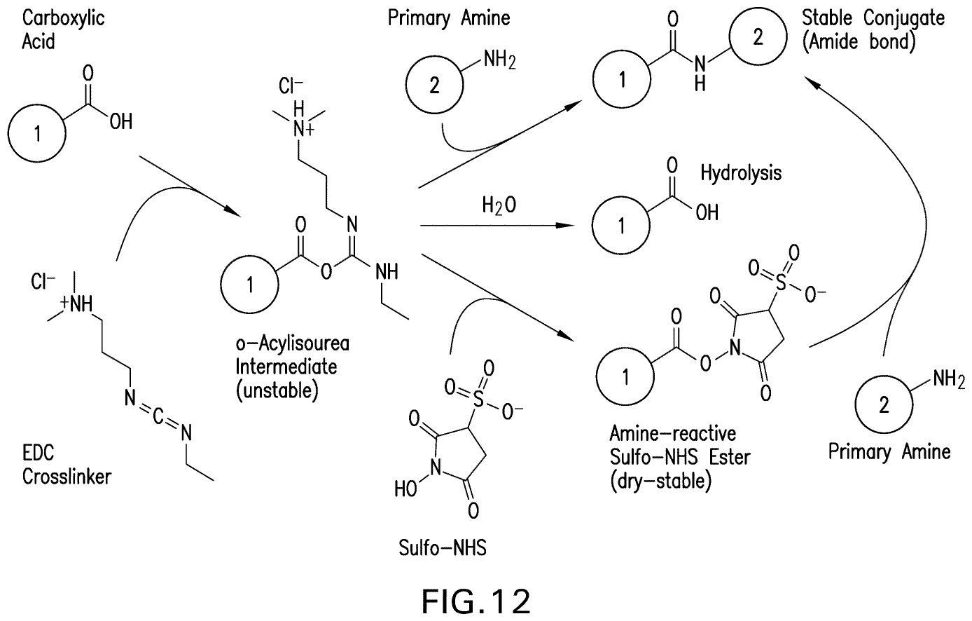

Three different capture particles were selected: 2.8 μm Protein A dynabeads, 2.8 μm Carboxylated dynabeads, and carboxylated 0.28 μm magnetic gold nanoshells. With a carboxylated surface instead of a Protein A surface, it is advantageously possible to utilize covalent chemistry to form an amide bond between the carboxyl group and the protein via EDC crosslinking to yield a more stable and reproducible functionalized capture reagent (FIG. 12). Specifically, the beads can be coated with carboxylic acid functional groups activated with EDC and sulfo-NHS to react with amines on antibody to form a stable amide bond.

Similarly, detection reagents can also be prepared utilizing covalent chemistry. Two different detection reagents were selected: NHS-ester fluorophores and mixed-isomer TRITC As the fluorophore is designed to function similarly to the HRP-conjugated antibodies in ELISA efforts, only unconjugated antibodies were used for functionalization of the capture and detector molecules.

Initial conjugations were performed using antibody 3 (Ab-3) with Protein A dynabeads (PrA) and magnetic gold nanoshells (mGNS). PrA conjugations were performed following vendor-recommended protocol, while mGNS conjugations were performed utilizing the covalent conjugation process via EDC-NHS amide bond formation.

Once magnetic capture reagents had been prepared, antibody 2 (Ab-2) was covalently conjugated to Alexafluor555 fluorophore. Conjugation was performed according to vendor-recommended protocol with a yield of <40%.

### Fluorophore Conjugate Optimization

Antibodies used during initial conjugation efforts had been supplied in 0.09% sodium azide. The low yield obtained during the first pass of antibody conjugation to the AlexaFluor555 can be due to the presence of this preservative which can interfere with conjugation by reacting with the carboxyl-NHS intermediate in the amide-bond formation process, instead of the antibody.

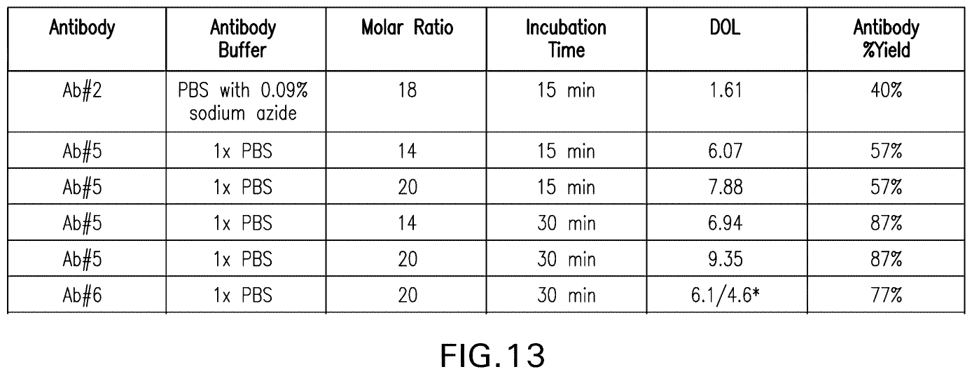

The purified antibodies were conjugated to the fluorophore following the vendor-provided protocol, with an increase in the incubation step time from 15 minutes to 30 minutes. The use of purified antibody in conjunction with an increased incubation allowed for yields up to 87% (FIG. 13).

With increased yields, additional platform testing was evaluated using the K12 negative strain and 1.00E+07 CFU/mL Escherichia coli O157:H7. Regardless of concentration of the conjugated fluorophore, fluorescent signal was unable to be observed using the detection apparatus.

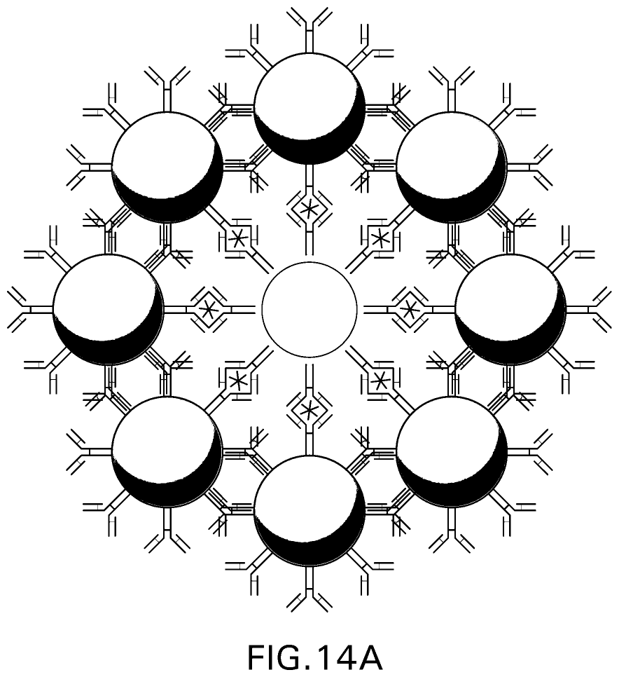

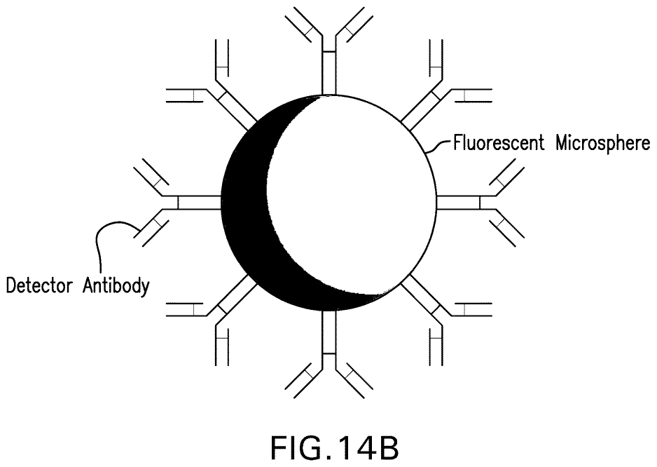

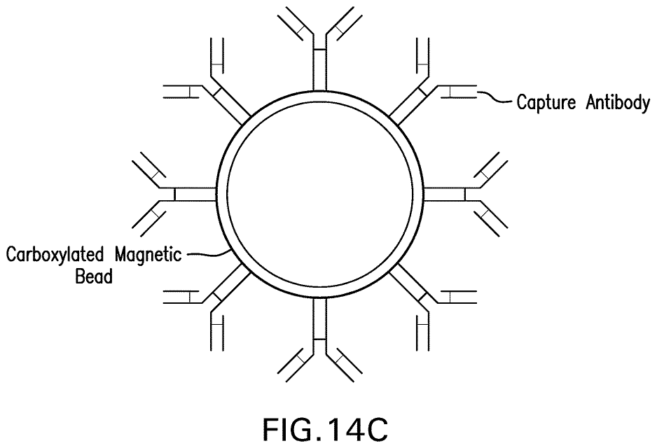

Due to the limitations on the degree of labeling (DoL), it is unlikely that there would be sufficient quantity of fluorophore per binding event, resulting in insufficient fluorescence for the platform to detect. To increase the amount of fluorescence per binding event, carboxylated fluorophore-dyed latex microspheres were assessed. The microspheres are impregnated with a substantial amount of fluorophore to allow for a larger signal to be produced in a sandwich type of assay (FIGS. 14A, 14B, and 14C).

### Conjugation and Evaluation of Fluorescent Microspheres

Fluorescent microspheres with various excitation and emission wavelength characteristics were assessed. Of the identified microspheres, fluorescent microspheres were predominantly used during assay feasibility on the detection apparatus. Fluorescence microspheres were selected for their 365 nm/610 nm excitation and emission. Fluorescence microspheres were selected for their compatibility with the blue illumination setting equipped on the detection apparatus.

Fluorescent microsphere conjugates were evaluated with mGNS, PrA, and carboxylated dynabead (COOH) conjugates using K12 and Escherichia coli O157:H7 samples at high concentrations for initial assay functionality evaluation. Test optimization was performed by varying the volume ratios of the assay conjugates. A wash step was incorporated following incubation of conjugates and sample to mitigate residual material from impacting assay readouts.

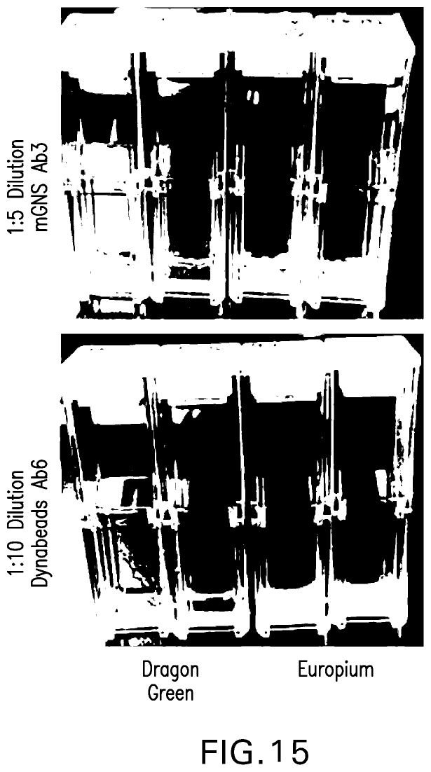

Initial evaluation of green-fluorescent conjugate was unsuccessful in yielding signal with positive test conditions, however clear signal with positive sample was visible using europium fluorescent conjugate in test solutions containing both mGNS and COOH conjugates (FIG. 15). The signal was clearly distinguishable from insignificant signal observed in negative tests.

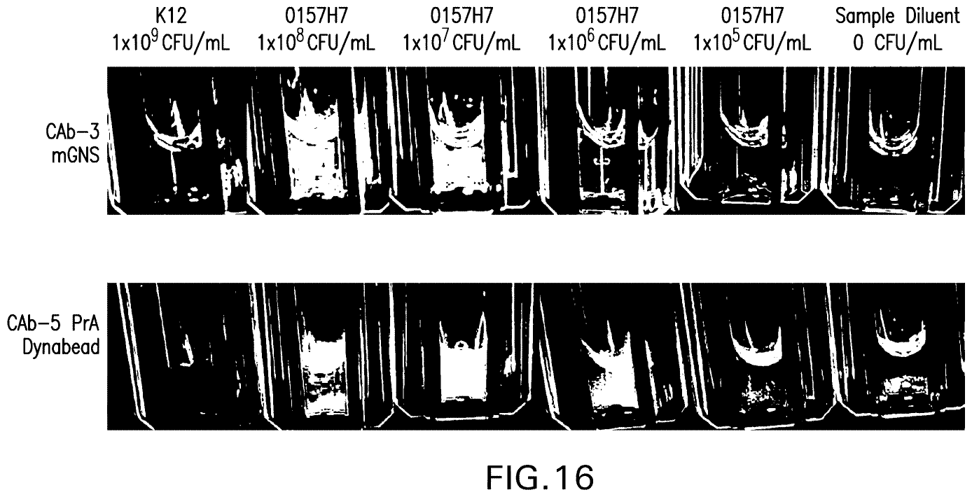

Escherichia coli O157:H7 bacteria was evaluated at various concentrations ranging from 1.00E+08 CFU to 1.00E+05 CFU/mL with both magnetic conjugates using europium microsphere detection conjugate. Fluorescence was visually distinguishable from negative (1.00E+09 CFU/mL K12) at positive bacteria concentrations as low as 1.00E+06 CFU/mL with both magnetic capture reagents (FIG. 16).

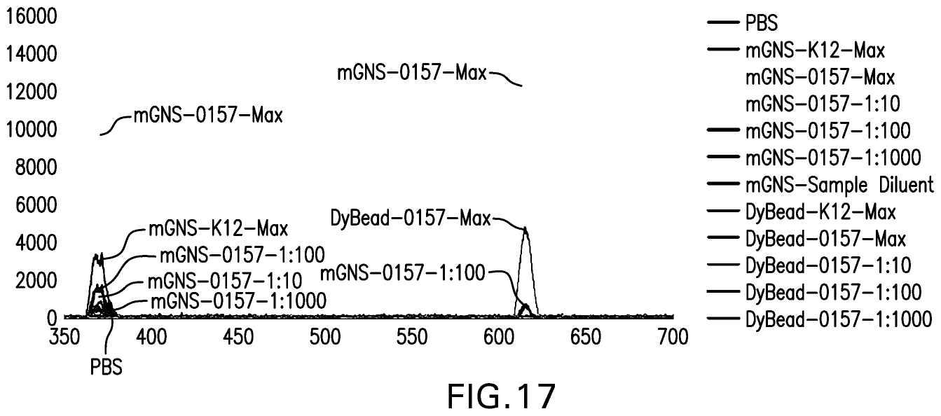

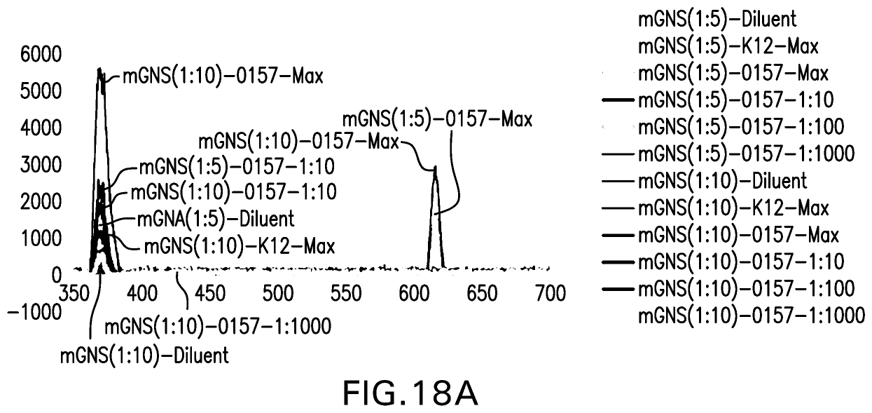

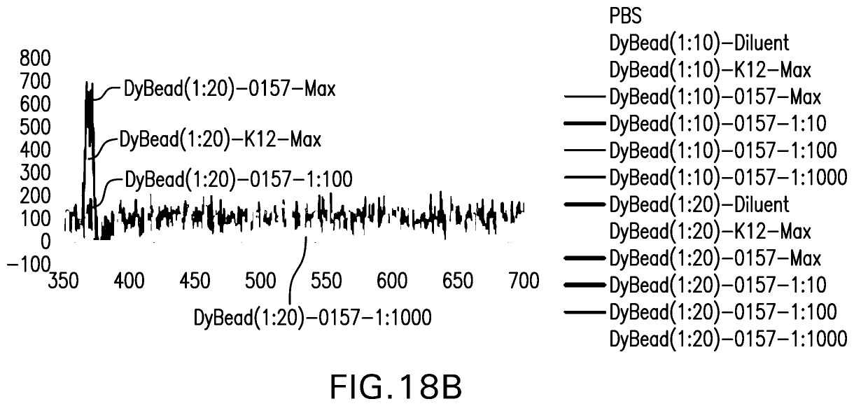

Thus far, signal with fluorescence was only visualized using a UV LED light. To facilitate use of fluorescence microspheres in the final test system, a detection apparatus equipped with a 365 nm LED light was used. The platform was successful in detecting signal with mGNS conjugates as low as 1.00E+06 CFU/mL but was unable to detect signal with samples containing COOH conjugates (FIGS. 17, 18A, and 18B). Lack of fluorescence in COOH samples was accompanied by a notably lower intensity scatter peak, suggesting the particle concentration may be too high in COOH samples.

### Mode Selections

Following successful dose response with Escherichia coli O157:H7, optimal conditions were considered and evaluated. The sandwich assay is comprised of four key components: a magnetic capture reagent and a magnetic conjugate antibody forming a capture moiety and a fluorescent detection reagent and a fluorescent conjugate antibody forming the detection moiety.

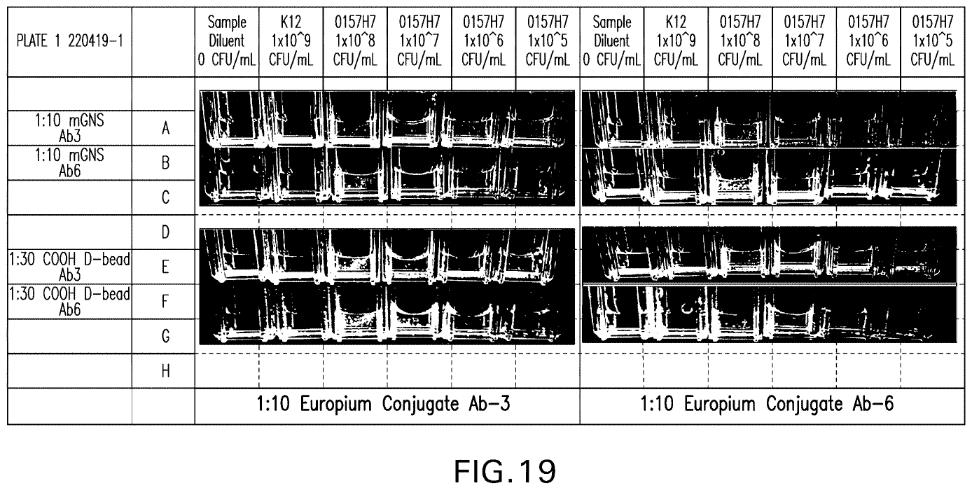

Two magnetic particles were selected based on their compatibility for covalent conjugation techniques: mGNS and COOH dynabeads. Both magnetic particles had exhibited functionality with Ab-3 and Ab-6 in sandwich assays utilizing europium microspheres. A test matrix was performed with mGNS, COOH, and europium microspheres conjugated with Ab-3 as well as respective conjugates prepared with Ab-6. The testing further allowed evaluation of higher dilution conditions with COOH conjugates. The results shown in FIG. 19 suggest optimal fluorescence with europium microspheres conjugated to Ab-3. However, no significant difference was observed with the magnetic conjugates.

To move forward, Ab-6 was selected for magnetic conjugates. Due to commercial availability and scalability, COOH dynabeads were selected as the ideal magnetic particle for the assay. Although mGNS required less material, the product is not as readily available but may be considered for later optimization efforts.

Fluorescence conjugated to Ab-3 appeared to be optimal, but additional evaluation of fluorescent microspheres was carried out. The detection apparatus has been optimized to evaluate fluorescence in the visible spectrum and it was believed that having a functional fluorescent microsphere would be ideal for the first generation of this particular assay.

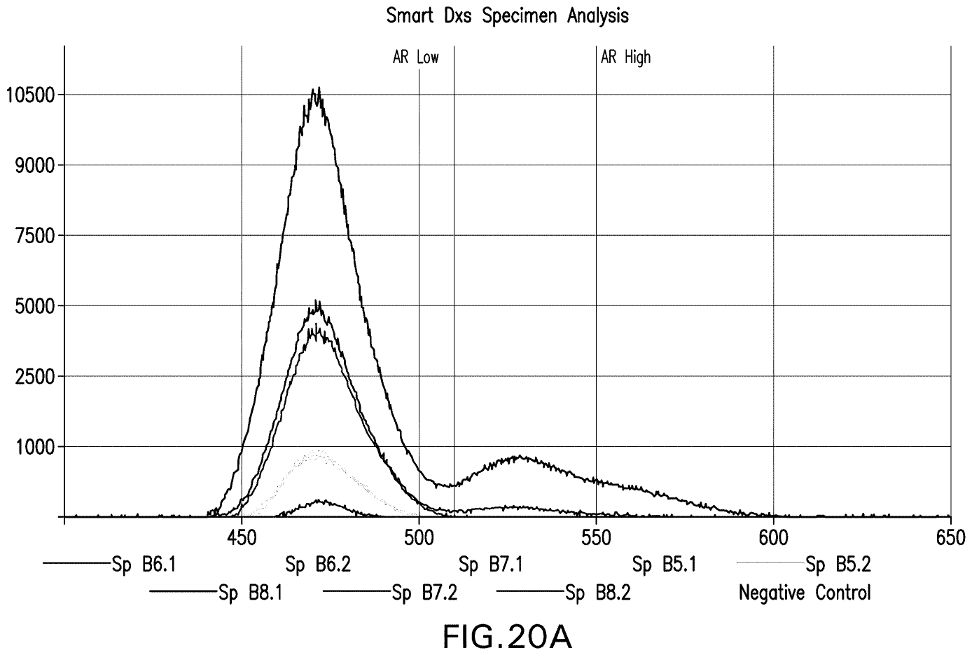

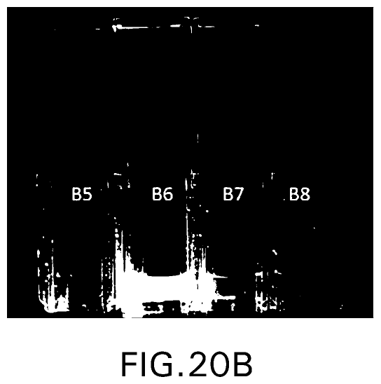

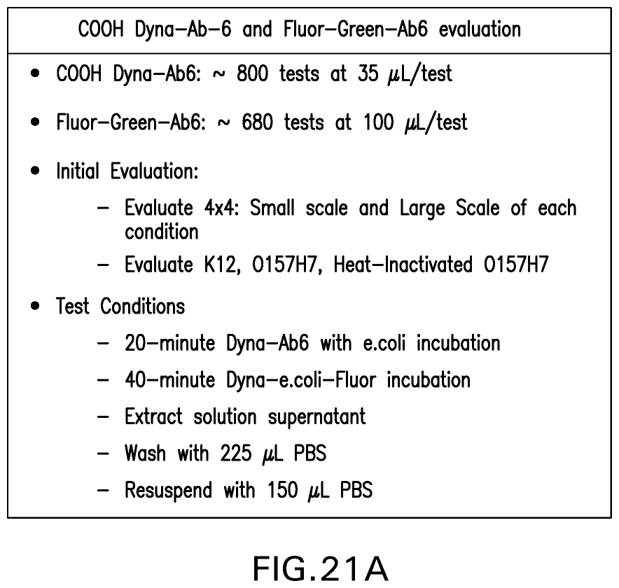

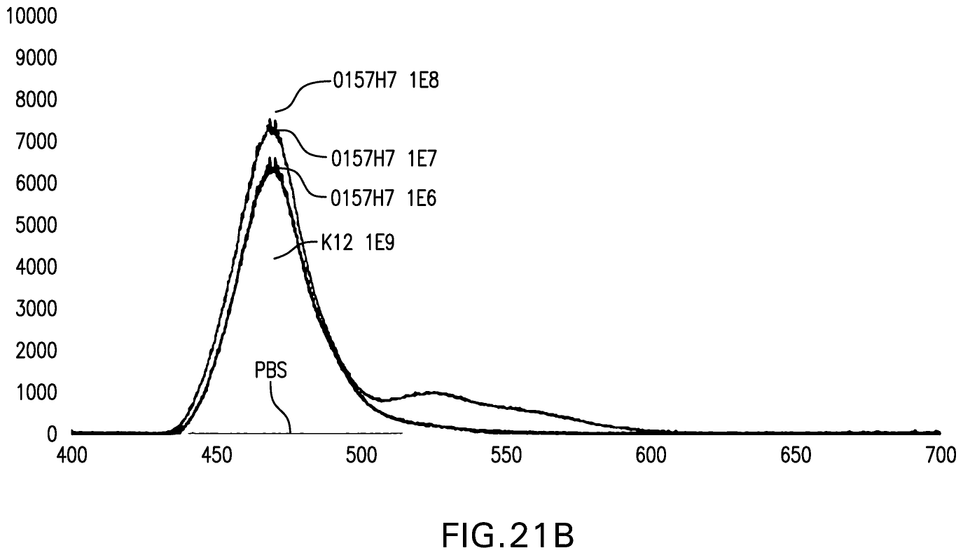

Fluorescent microspheres were conjugated with Ab-6, following a hybridized conjugation. The microspheres were evaluated with COOH magnetic dynabeads conjugated to Ab-6. Testing was successful in achieving positive signal with the original detection-apparatus blue illumination settings (FIGS. 20A and 20B).

The fluorescent conjugates elicited visually stronger fluorescence, but a test system utilizing a single antibody (Ab-6) and fluorescent microspheres compatible with the original platform design conditions prompted selection of green-fluorescent microsphere as the detection particle.

### Platform Training

Platform training is the process of running a high volume of samples so an algorithm can accurately predict qualitative and/or quantitative results. Non-limiting aspects of platform training are described below.

Antibody 6 (Ab-6) was obtained in large quantities and purified into 1x PBS using spin column filtration. The purified antibody was used to prepare magnetic and fluorescent conjugates with carboxylated dynabeads (COOH) and green-fluorescent microspheres, respectively. The conjugates were prepared on scales that would allow sufficient testing for the detection-apparatus training and analytical sensitivity evaluation.

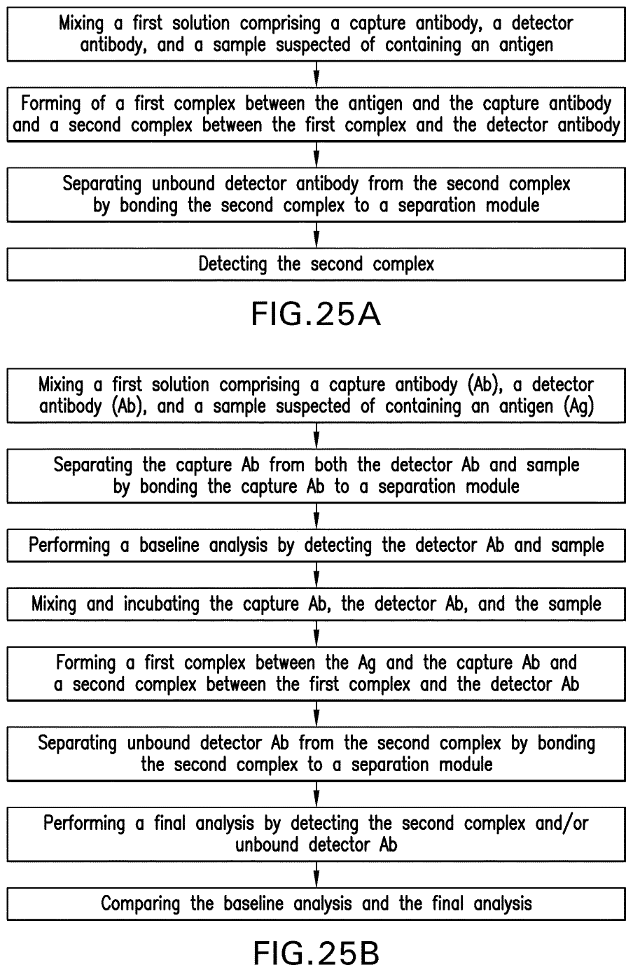

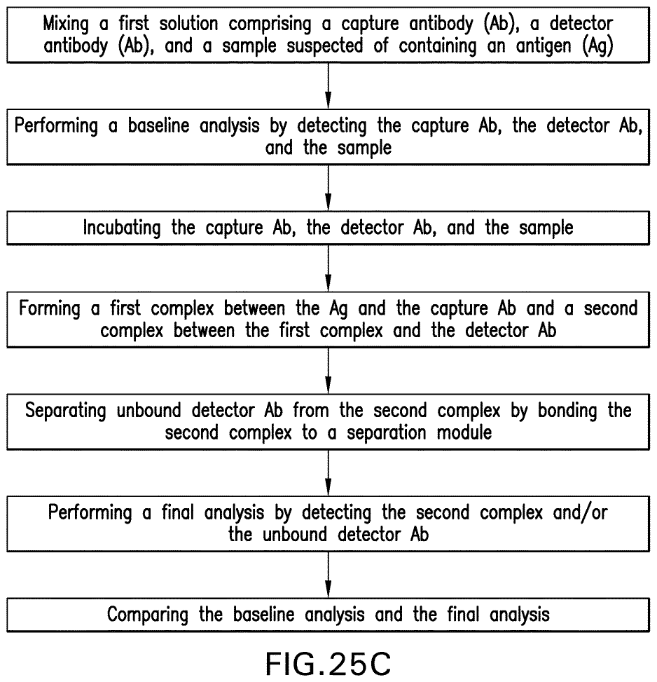

The large-scale conjugations were evaluated against respective small-scale conjugations in a 4×4 test matrix. The results from this test matrix yielded improved results with large-scale conjugation pairs (FIGS. 25A and 25B). These results were made possible through increasing the volume of the fluorescent conjugate from 50 μL to 100 μL while following the currently established protocol requiring a 20-minute incubation of the capture conjugate with the sample, followed by the introduction of the fluorescent detector conjugate and a 40-minute incubation.

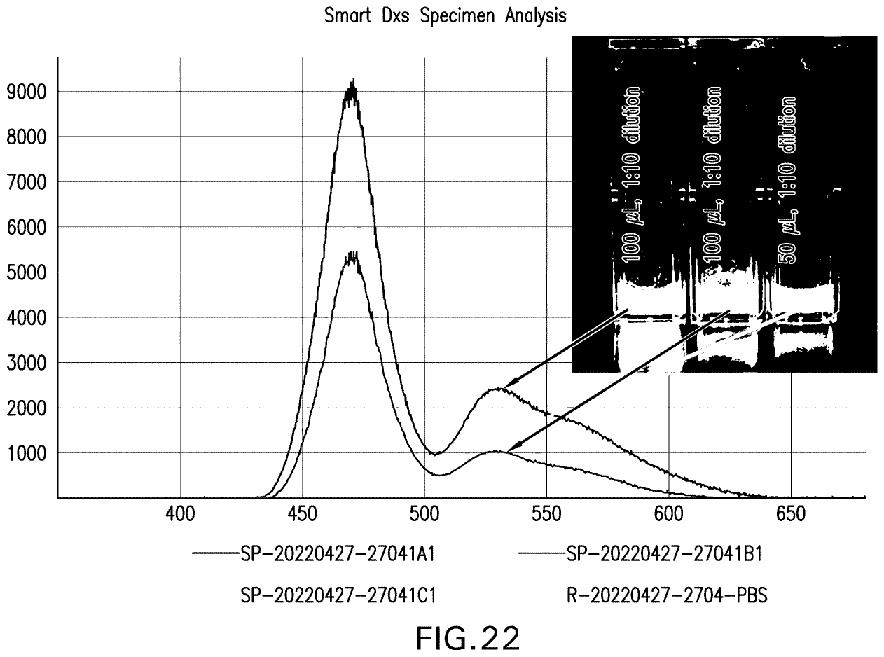

The increase in detection reagent volume per test reduced the potential quantity of tests that could be run with the same lot of material. To determine if less volume could be successfully employed, testing was performed with green-fluorescent detection reagent using 100 μL of the standard 1:10 dilution, 100 μL of a 1:15 dilution, and 50 μL of a 1:10 dilution. The tests were carried out with optimized magnetic capture conjugate, utilizing two incubation steps: 20-minute incubation of capture conjugate with sample followed by a 60-minute incubation of the detection conjugate with the capture conjugate and sample. A significant increase in signal with 1.00E+08 and 1.00E+07 CFU/mL Escherichia coli O157 :H7 samples and no signal with 1.00E+09 CFU/mL K12 with the 20-minute extension of the second incubation were observed (FIG. 22). Further, the reduction of detection reagent volume to 50 μL was observed to yield signal of near-equal intensity to the signal observed with 100 μL of detection reagent with the 40-minute long second incubation step.

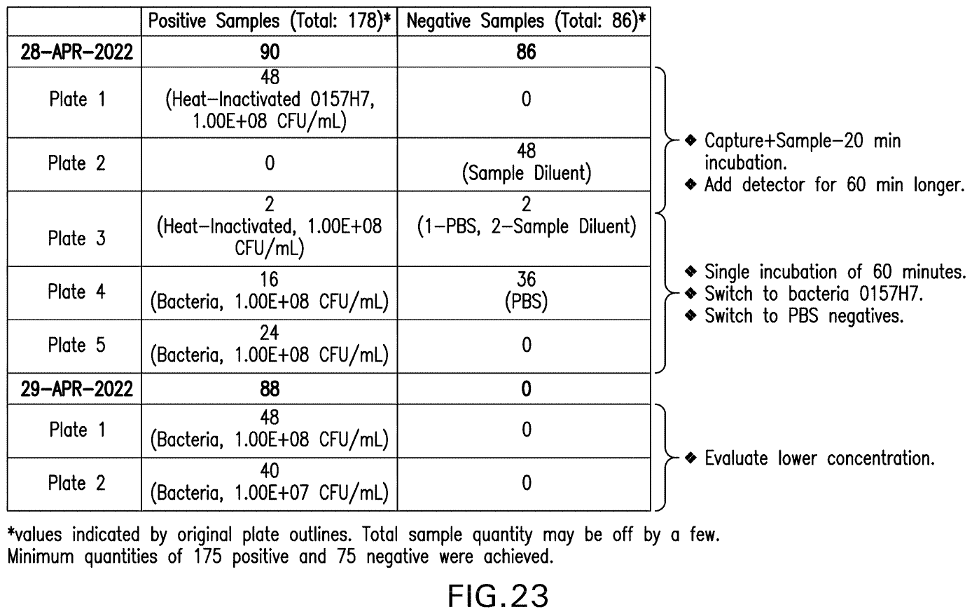

The results acquired from volume and dilution experiments were utilized to optimize the platform training testing, in which over 175 positive samples (Escherichia coli O157:H7) and over 75 negative samples (PBS, sample diluent) were evaluated with capture conjugate (COOH diluted 1:30) and detection conjugate (green microspheres diluted 1:10). 50 positive tests utilized 1.00E+08 CFU/mL heat-inactivated Escherichia coli O157:H7. The remaining positive tests were carried out with cultured Escherichia coli O 157:H7 at 1.00E+08 and 1.00E+07 CFU/mL. Initial negative tests were carried out using sample diluent, but the final 36 negative tests utilized 1x PBS as a control for what a 3rd party validation run would utilize. After running the first 96 samples, the 20-minute primary incubation step was removed, and all tests were successfully executed using a single 60-minute co-incubation of capture, sample, and detector (FIG. 23).

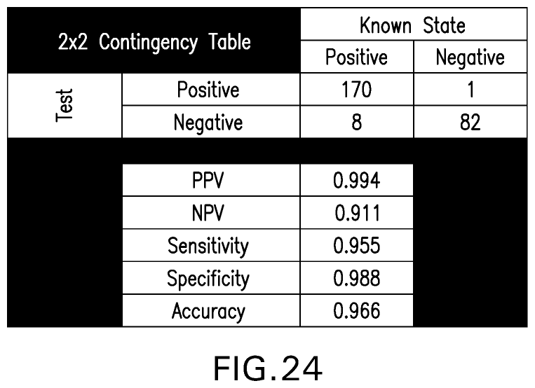

Throughout training, several negative samples yielded “presence” results due to the scatter peak tail overlapping the analysis region of the detection apparatus software. However, these false positive results were easily resolved by shortening the analysis region to only detect within the range of fluorescence. There were eight positive samples that yielded “absence” results during the training procedure. These false negatives all appeared to be in a single column of a given test plate, suggesting there was an operator error. Even with the inclusion of the false negative results, the training data yielded >95% sensitivity using the detection apparatus Beta-010 test platform (FIG. 24).

Sensitivity of >95% was successfully observed with Escherichia coli O157 :H7 samples as low as 1.00E+07 CFU/mL. The signal acquired with these samples was reasonably strong and suggestive of a lower limit of detection. Operators evaluated the analytical sensitivity via a series of serial dilutions-observing signal in samples as low as 4.00E+06 CFU/mL. When confirming this analytical sensitivity, 15 samples were evaluated at 1.00E+07 CFU/mL, 15 samples were evaluated at 4.00E+06 CFU/mL, and 15 samples were evaluated at 1.00E+06 CFU/mL. PBS was used as a negative control in five replicates. The confirmation testing resulted in positive signal observed with all 45 samples containing Escherichia coli O157:H7 and no signal observed with the 5 PBS replicates.

An additional round of training was performed on Platform Beta-012. This platform had previously yielded stronger signal intensity and, thus far, only had a small quantity of testing performed during training. 50 positive samples (1.00E+07 CFU/mL Escherichia coli O157:H7) and 15 negative samples (1x PBS) were evaluated following established testing protocols finalized in Platform beta-010 training. The Beta-012 detection apparatus platform yielded 100% accuracy in identifying presence vs absence on the respective positive and negative samples.

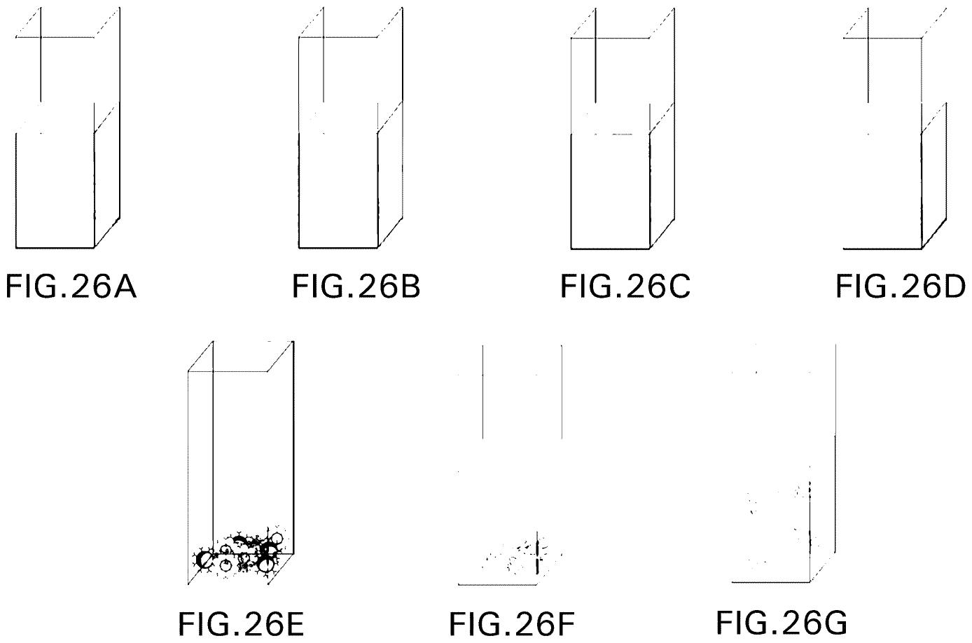

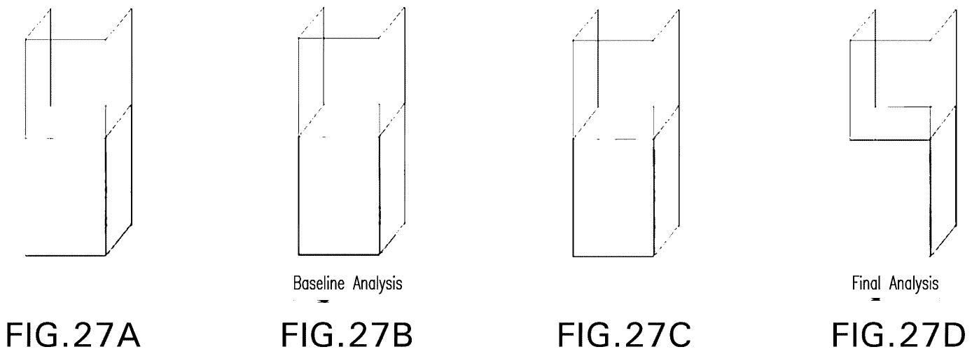

Optimized conditions established for the assay include the prepared reagents required for testing in addition to the optimized testing protocol. A flow chart and a schematic representation of different steps of assays are shown in FIG. 25, FIGS. 26A-26G, and FIGS. 27A-27D.

The contents of all cited references (including literature references, patents, patent applications, and websites) that maybe cited throughout this application are hereby expressly incorporated by reference in their entirety for any purpose, as are the references cited therein.

Other embodiments are within the following claims.

What is claimed:

1. A method of detecting an antigen, the method comprising:
A:
mixing a solution comprising a capture antibody, a detector antibody, and a sample suspected of containing the antigen, wherein:
the capture antibody is linked to a capture moiety by a functional group and specifically binds to the antigen in conditions sufficient to form a first complex between the antigen and the capture antibody; and
the detector antibody is linked to a detection moiety and specifically binds to the antigen in conditions sufficient to form a second complex between the first complex and the detector antibody, wherein the detection moiety is a light-emitting microsphere;
separating unbound detector antibody linked to the detection moiety from the second complex; and
detecting the light-emitting microspheres on the second complex by spectroscopy, thereby detecting the antigen;
B:
mixing a solution comprising a capture antibody, a detector antibody, and a sample suspected of containing the antigen, wherein the capture antibody is linked to a capture moiety by a functional group, and the detector antibody is linked to a detection moiety, wherein the detection moiety is a light-emitting microsphere;
separating the capture antibody from both the detector antibody and sample;
performing a baseline analysis by detecting the light-emitting microspheres from the detector antibody by spectroscopy;
mixing and incubating the capture antibody, the detector antibody, and the sample, wherein the capture antibody specifically binds to the antigen in conditions sufficient to form a first complex between the antigen and the capture antibody, and wherein the detector antibody specifically binds to the antigen in conditions sufficient to form a second complex between the first complex and the detector antibody;
separating unbound detector antibody from the second complex;
performing a final analysis by detecting the light-emitting microspheres on the second complex and/or the light-emitting microspheres from the unbound detector antibody by spectroscopy; and
comparing the baseline analysis and the final analysis,
thereby detecting the antigen; or
C:
mixing a solution comprising a capture antibody, a detector antibody, and a sample suspected of containing the antigen, wherein the capture antibody is linked to a capture moiety by a functional group, and the detector antibody is linked to a detection moiety, wherein the detection moiety is a light-emitting microsphere;
performing a baseline analysis by detecting the light-emitting microspheres from the detector antibody of the mixture by spectroscopy;
incubating the capture antibody, the detector antibody, and the sample, wherein the capture antibody specifically binds to the antigen in conditions sufficient to form a first complex between the antigen and the capture antibody, and wherein the detector antibody specifically binds to the antigen in conditions sufficient to form a second complex between the first complex and the detector antibody;
separating unbound detector antibody from the second complex;
performing a final analysis by detecting the light-emitting microspheres on the second complex and/or the light-emitting microspheres from the unbound detector antibody by spectroscopy; and
comparing the baseline analysis and the final analysis,
thereby detecting the antigen.

2. The method of claim 1, wherein the function group is a carboxylic functional group.

3. The method of claim 1 or 2, wherein the light-emitting microsphere is a fluorescent microsphere.

4. The method of claim 3, wherein the fluorescent microsphere comprises a fluorescent dye.

5. The method of claim 4, wherein the fluorescent dye is an Alexa fluorescent dye.

6. The method of claim 4 or 5, wherein the fluorescent dye is encapsuled inside the light-emitting microsphere.

7. The method of any one of claims 4-6, wherein the light-emitting microsphere comprises a polymer encapsulating the fluorescent dye.

8. The method of any one of claims 1-7, wherein the light-emitting microsphere has an average diameter of between about 20 nm and about 5000 nm, about 100 nm and about 2000 nm, about 100 nm and about 1000 nm, or about 100 nm and about 600 nm.

9. The method of any one of claims 1-8, wherein the light-emitting microsphere has an average diameter of about 167 nm, about 200 nm, about 300 nm, or about 500 nm.

10. The method of any one of claims 1-9, wherein the light-emitting microsphere has an excitation wavelength between about 350 nm to about 850 nm.

11. The method of any one of claims 1-10, wherein the light-emitting microsphere emits light at a wavelength between about 350 nm to about 700 nm.

12. The method of any one of claims 1-11, wherein the detector antibody is linked to the detection moiety by a second functional group.

13. The method of claim 12, wherein the second functional group is a carboxylic functional group.

14. The method of any one of claims 1-13, wherein the antigen is located on the surface of a microbe.

15. The method of claim 14, wherein the microbe is a foodborne bacteria, an environment-borne bacteria, or a bloodborne bacteria.

16. The method of claim 14 or 15, wherein the microorganism is a bacterium.

17. The method of claim 13, wherein the bacterium is an Escherichia coli or Salmonella spp.

18. The method of claim 17, wherein the Escherichia coli is an Escherichia coli O157:H7 strain.

19. The method of any one of claims 1-18, wherein the capture moiety comprises a metallic particle, a polymeric particle, or a combination thereof.

20. The method of claim 19, wherein the metallic particle is a magnetic particle.

21. The method of claim 20, wherein the magnetic particle has an average diameter of between about 0.005 μm and about 4.5 μm, about 0.1 μm and about 4.5 μm, about 1.0 μm and about 4.0 μm, or about 2.0 μm and about 3.0 μm.

22. The method of claim 20 or 21, wherein the magnetic particle has an average diameter of about 2.8 μm.

23. The method of claim 20, wherein the magnetic particle has an average diameter of between about 0.1 μm and about 2.0 μm, or about 0.1 μm and about 0.5 μm.

24. The method of any one of claims 20-23, wherein the magnetic particle comprises carboxylic acid groups.

25. The method of any one of claims 1-24, comprising separating unbound detector antibody linked to the detection moiety from the second complex by bounding the second complex to a separation module.

26. The method of claim 25, wherein the second complex is bounded to the separation module using a magnetic field.

27. The method of any one of claims 1-26, wherein the light-emitting microsphere on the second complex are detected by spectroscopy in solution.

28. The method of any one of claims 1-27, wherein the detection is qualitative.

29. The method of any one of claims 1-27, wherein the detection is quantitative.

30. The method of any one of claims 10-29, wherein:
the microorganism is present at a concentration of at least about 1.00E+05 CFU/mL, at least about 1.00E+06 CFU/mL, or at least 1.00E+07 CFU/mL; or
the microorganism is present at a concentration of about 1.00E+05 CFU/mL to about 1.00E+10 CFU/mL, about 1.00E+5 CFU/mL to about 1.00E+9 CFU/mL, about 1.00E+5 CFU/mL to about 1.00E+8 CFU/mL, about 1.00E+5 CFU/mL to about 1.00E+7 CFU/mL, or about 1.00E+5 CFU/mL to about 1.00E+6 CFU/mL.

31. The method of any one of claims 1-30, wherein one or more of the method steps are perform in one or more sample containers, optionally wherein one or more of the method steps are perform in one sample container.

32. The method of claim 31, wherein the sample container has a volume in a range of about 0.5 mL to about 15 mL, about 1.0 mL to about 10, or about 2 mL to about 5 mL.

33. The method of any one of claims 1-32, wherein the step of incubating comprises heating.

34. The method of any one of claims 1-33, wherein one or more of the method steps are perform automatically and/or manually.

35. An apparatus for the detection of an antigen, comprising:
a solution comprising a capture antibody, a detector antibody, and a sample suspected of containing the antigen, wherein:
the capture antibody is linked to a capture moiety by a functional group and specifically binds to the antigen in conditions sufficient to form a first complex between the antigen and the capture antibody; and
the detector antibody is linked to a detection moiety and specifically binds to the antigen in conditions sufficient to form a second complex between the first complex and the detector antibody, wherein the detection moiety is a light-emitting microsphere;
a plurality of sample containers, wherein each sample container is configured for:
receiving the solution comprising the capture antibody, the detector antibody, and the sample suspected of containing the antigen;
receiving illumination at a first wavelength range from a light source; and
transmitting radiation from the detection moiety; and
an optical instrument comprising:
a plurality of sample container receiving positions, each for receiving a respective one of the plurality of sample containers;
the light source adjacent to at least one of the sample containers receiving positions for providing illumination at the first wavelength range and in a first illumination direction through a respective sample container once disposed within the respective sample container receiving position for causing radiation from the detection moiety bound within the detection platform;
a carousel with one or more repositionable mirrors for focusing the illumination from the light source; and
a photodetector disposed adjacent to the sample container receiving positions for selectively detecting the energy of light radiated from the detection moiety within the respective sample container in response to illumination from the light source and for generating an electrical signal corresponding to the detected energy of the radiated light in response thereto.

36. The apparatus of claim 35, wherein the optical instrument further comprises an amplifier for amplifying the electrical signal output by the photodetector in response to detecting the energy of the radiated light.

37. The apparatus of claim 35 or 36, wherein the optical instrument further comprises a digital decoder for converting the detected energy of the radiated light from the respective sample container, represented by the electrical signal output by the photodetector and optionally amplified by the amplifier, to a detectable signal for the detection of the antigen.

38. The apparatus of claim 37, wherein the detectable signal is use as a measure of the concentration of the antigen bound within the second complex, the detected energy of the radiated light being proportional to a measure of detection moiety present in the respective sample container and thus to a measure of antigen concentration bound by the capture antibody.

39. An apparatus for geolocating a source of potential foodborne, environment-borne, and/or bloodborne bacteria, comprising:
a solution comprising a capture antibody, a detector antibody, and a sample suspected of containing the bacteria, wherein:
an antigen is located on the surface of the bacteria;
the capture antibody is linked to a capture moiety by a functional group and specifically binds to the antigen in conditions sufficient to form a first complex between the antigen and the capture antibody; and
the detector antibody is linked to a detection moiety and specifically binds to the antigen in conditions sufficient to form a second complex between the first complex and the detector antibody, wherein the detection moiety is a light-emitting microsphere;
one or more sample containers;
an optical instrument; and
a global positioning system (GPS).
